(19) United States
(12) Patent Application Publication
(10) Pub. No.: US 2004/0202697 A1
(43) Pub. Date: Oct. 14, 2004

(54) Use of proteolytic enzymes to increase feed utilization in ruminant diets

(76) Inventors: Karen A. Beauchemin, Lethbridge (CA); Dario Colombatto, Gualeguaychu (AR)

Correspondence Address: GREENLEE WINNER AND SULLIVAN P C, 5370 MANHATTAN CIRCLE, SUITE 201, BOULDER, CO, 80303, US

(21) Appl. No.: 10/778,925

(22) Filed: Feb. 13, 2004

Related U.S. Application Data
(60) Provisional application No. 60/452,737, filed on Mar. 7, 2003.

(51) Int. Cl. A61K 38/48; A23K 1/165

(52) U.S. Cl. 424/442; 424/94.63

(57) ABSTRACT

The invention provides a method of increasing fiber digestion in ruminants by providing a feed additive or feed composition comprising at least one protease. Specifically, the invention provides a method of increasing digestibility of a forage or a grain feed comprising providing at least one protease; providing a forage or a grain feed suitable for a ruminant animal; applying the protease to the forage or the grain feed; and administering the composition to the animal, whereby an increase in digestibility is effected. The invention further extends to feed additives and feed compositions comprising proteases, preparations and uses thereof.

### CROSS REFERENCE TO RELATED APPLICATIONS

This application claims priority from U.S. Provisional Patent Application Ser. No. 60/452,737 filed Mar. 7, 2003, which is incorporated by reference herein to the extent that there is no inconsistency with the present disclosure.

### FIELD OF THE INVENTION

This invention relates to a method of increasing fiber digestion in ruminant by providing a feed additive or feed composition comprising proteases.

### BACKGROUND OF THE INVENTION

Ruminants are mammals which possess a special digestive organ, the rumen, within which efficient digestion of plant fiber occurs through the activity of anaerobic microorganisms (bacteria, fungi, protozoa). Ruminants subsist primarily on plant fiber derived from grasses and legumes, with the plant fiber consisting of insoluble polysaccharides, particularly cellulose and hemicellulose. While most mammals lack the enzymes necessary to digest such polysaccharides, ruminants rely upon microorganisms as digestive agents. While food remains in the rumen, cellulolytic microorganisms hydrolyze cellulose to the disaccharide cellobiose and to free glucose units. The released glucose then undergoes a bacterial fermentation with the production of volatile fatty acids (i.e., acetic, propionic and butyric) and gases (carbon dioxide and methane). The volatile fatty acids travel across the rumen wall to the bloodstream and are oxidized by the ruminant as its main source of energy. Carbon dioxide and methane are removed by eructation to the atmosphere. In addition, the microorganisms synthesize amino acids and vitamins.

Although the rumen is an efficient mechanism for digestion this process is slow and often incomplete, particularly with higher fiber feeds. This inefficiency leads to increased cost of livestock production, increased use of feed resources, and increased environmental impact of ruminant production. Approaches to increase the extent of utilization of fiber by ruminants using physical treatments (e.g., grinding steam treatment, pelleting, etc.) or chemical treatments (e.g., alkalis, ammonia, urea, ozone, etc.) can be undesirable due to expense and danger posed to humans and the environment.

Alternative treatments, such as biological catalysts or enzymes to expedite feed digestion in the rumen, are desirable. Increased feed digestion enhances the productivity of the animal and can reduce the costs of production. In addition, it may also reduce the impact of livestock production on the environment by reducing the amount of manure excreted by the animals and by reducing the quantity of feed needed to obtain a specific level of production.

Enzymes are proteins which accelerate or catalyze biological reactions, and are secreted by microorganisms (mainly fungi or bacteria). Enzymes which degrade the plant cell wall or “fiber” are collectively termed cellulases and hemicellulases, depending on the fiber fraction (cellulose or hemicellulose) which they degrade. Cellulases and hemicellulases are used widely in the textile, food, brewing, detergent, and feed industries. In animal nutrition, they are used in the monogastric (poultry and swine) industry; however, their use in ruminants remains undeveloped.

Early research using enzymes in ruminant diets was inconclusive due to poor characterization of the enzymes used. Further, this use was viewed with skepticism since it was believed that unprotected enzymes would be inactivated rapidly in the rumen due to high proteolytic activity. In addition, since the ruminal microbes themselves degrade the feed by secreting enzymes of the same type of those being added, it was thought that supplemental enzymes would not have any positive effect. However, research using newer and better characterized enzyme mixtures have demonstrated not only that these enzymes are capable of resisting the rumen environment for a time long enough to alter digestion, but also that addition of specific enzyme mixtures increases feed digestion and animal performance (i.e., feedlot cattle and dairy cows). U.S. Pat. No. 5,720,971 to Beauchemin et al. teaches fiber-digesting enzyme supplements comprising mixtures of cellulases and xylanases in certain preferred ratios and levels, and use thereof for increasing the digestibility of legume forages and grain feed for ruminants.

Traditional ruminant research has focused on cellulases and hemicellulases, and occasionally on pectinases and amylases. In contrast, the use of proteases in ruminant diets has been ignored. A possible reason is that excessive protein degradation in the rumen is considered as nutritionally inefficient, as it leads towards higher nitrogen losses from the animal and to an increase in pollution. However, the present invention surprisingly demonstrates that use of proteases in ruminant diets is effective and beneficial in increasing feed digestibility.

### SUMMARY OF THE INVENTION

The present invention broadly provides a method of increasing fiber digestion in ruminant by providing a feed additive or feed composition comprising at least one protease. Specifically, the invention provides a method of increasing digestibility of a forage or a grain feed comprising the steps of providing at least one protease; providing a forage or a grain feed suitable for a ruminant animal; applying the protease to the forage or the grain feed to form a feed composition; and administering the composition to the animal, whereby an increase in digestibility is effected.

In another aspect, the invention provides a method of feeding a ruminant animal comprising the steps of providing at least one protease; providing a forage or a grain feed; applying the protease to the forage or the grain feed to form a feed composition; and administering the composition to the animal, whereby an increase in digestibility is effected.

In another aspect, the invention provides a method of treating a forage or a grain feed to increase digestibility comprising the steps of providing at least one protease; providing a forage or grain feed suitable for a ruminant animal; applying the protease to the forage or the grain feed to form a feed composition; and administering the composition to the animal, whereby an increase in digestibility is effected.

In another aspect, the invention provides a method of producing a feed additive comprising the steps of providing at least one protease; mixing the protease with one or more inert or active ingredients to form the feed additive; and feeding the feed additive to a ruminant animal or adding the feed additive to a forage or a grain feed for the animal, whereby an increase in digestibility is effected.

In another aspect, the invention provides a method of producing a feed composition for feeding to a ruminant animal comprising the steps of providing at least one protease; providing a forage or a grain feed; and applying the protease to the forage or the grain feed to form the composition, whereby an increase in digestibility is effected.

In another aspect, the invention provides a feed additive comprising at least one feed-grade protease in combination with one or more feed-grade inert or active ingredients, wherein the protease is included in an amount which increases digestibility of a forage or feed grain when applied to the forage or the feed grain and fed to an animal. In another aspect, the invention provides a feed composition for feeding to a ruminant animal comprising a forage or a grain feed in combination with at least one protease, whereby an increase in digestibility is effected.

In another aspect, the invention provides use of a protease for feeding a ruminant animal comprising the steps of providing at least one protease; providing a forage or a grain feed; applying the protease to the forage or the grain feed to form a feed composition; and administering the composition to the animal, whereby an increase in digestibility is effected.

In another aspect, the invention provides use of a protease for producing a feed additive comprising the steps of providing at least one protease; mixing the protease with one or more inert or active ingredients to form the feed additive; and feeding the feed additive to a ruminant animal or adding the feed additive to a forage or a grain feed for the animal, whereby an increase in digestibility is effected.

In yet another aspect, the invention provides use of a protease to produce a feed composition comprising the steps of providing at least one protease; providing a forage or a grain feed; and applying the protease to the forage or the grain feed to form the composition, whereby an increase in digestibility is effected.

As used herein and in the claims, the terms and phrases set out below have the following definitions.

“Rumen” means the largest compartment of the stomach of a ruminant.

“Ruminant” or “ruminants” is meant to include cattle, sheep, goats, camels, buffalo, deer, reindeer, caribou and elk which have a complex, multichambered stomach.

“Feed material” means a forage or grain feed or combination thereof.

“Grain feed” means the seeds of plants which are typically fed to ruminant animals which may or may not include the outer hull, pod or husk of the seed. Examples of grain feed include, without limitation, barley, wheat, corn, oats, sorghum, triticale, rye, and oilseeds.

“Forage” means the edible parts of plants, other than separated grains, which can provide feed for grazing animals or that can be harvested for feeding to ruminants.

“Legume forage” means the portion of a plant used as an animal feedstuff which is a dicotyledonous plant species that is a member of the botanical family Leguminosae. Examples include, without limitation, alfalfa, sainfoin, clovers and vetches. The term is meant to include forages comprising greater than 50% plant material from the Leguminosae family and the remaining plant material from other species.

“Mixed hay” means legume-grass mixed hay.

“Total mixed ration” abbreviated as “TMR” means a combination of two or more feed materials.

“Dry” means a feed material having a moisture content of less than 15% (w/w).

“Wet” means a feed material having a moisture content of greater than 15% (w/w).

“Dry matter” abbreviated as “DM” means the substance in a plant remaining after oven drying to constant weight.

“Organic matter” abbreviated as “OM” means the difference between the original feed composition and its ash content, determined by combustion at >500° C. for at least 3 h.

“Crude protein” abbreviated as “CP” means the estimate of protein content based on determination of total nitrogen (N) content×6.25.

“Neutral detergent fiber” abbreviated as “NDF” means the portion of feed which is insoluble in neutral detergent and is synonymous with cell wall constituents, excluding pectin.

“Acid detergent fiber” abbreviated as “ADF” means the insoluble residue following extraction of feed material with acid detergent, or cell wall constituents minus hemicellulose.

“Acid detergent lignin” abbreviated as “ADL” means the lignin or residue determined following extraction of ADF with concentrated sulphuric acid.

“Hemicellulose” means the polysaccharides associated with cellulose and lignin in the cell walls of plants, and includes glucans (apart from starch), mannans, xylans, arabinans or polyglucuronic or polygalacturonic acid. It is determined as the difference between NDF and ADF.

“Cellulose” means a carbohydrate comprised of glucose units which are linked by β-1,4 bonds.

“Apparent digestibility” means digestibility determined by animal feeding trials calculated as feed consumption minus excretion and expressed as a percentage of feed composition, but which does not account for endogenous excretion in the feces.

“True digestibility” means the actual digestibility or availability of feed, forage or nutrient as represented by the balance between intake and fecal loss of the same ingested material with endogenous excretions in feces accounted for. The term also reflects the in vitro digestibility.

“Volatile fatty acids” abbreviated a “VFA” are the endproducts of microbial fermentation in the rumen and provide energy to the host animal. VFA is meant to include, but is not limited to, acetic, propionic and butyric acids. Branched-chain volatile fatty acids are abbreviated as “BCVFA.”

“Enzyme mixture” means a combination of enzymes containing at least one protease.

“Cellulase” means an enzyme which digests cellulose to hexose units.

“Protease” or “proteases” means an enzyme which is capable of cleaving peptide bonds. The term is meant to include, without limitation, cysteine proteases, metalloproteases, aspartate proteases, and serine proteases.

“Protease activity” means the activity of proteases, namely the capacity to cleave peptide bonds, or protease activity as assayed at pH 6.0. 39° C. using 0.4% azocasein as substrate.

“Proteases as the major component” means that with the proteases as the major component, no other enzyme activity is required although other activities may be present.

“Serine protease” means an enzyme which is responsible for the catalysis of hydrolysis of peptide bonds, and which has an active serine residue in the active site. The term is meant to refer to trypsin-like and subtilisin-like types which have an identical spatial arrangement of catalytic His, Asp, and Ser but in quite different catalytic scaffolds.

“Subtilisin-like serine protease” means serine proteases whose catalytic activity is provided by a charge relay system similar to that of the trypsin family of serine proteases but which evolved by independent evolution. The sequence around the residues involved in the catalytic triad (aspartic acid, serine and histidine) are completely different from that of the analogous residues in the trypsin serine proteases and can be used as signatures specific to that category of proteases.

“Trypsin-like serine protease” is meant to include both mammalian enzymes such as trypsin, chymotrypsin, elastase, kallikren and thrombin having approximately 230 residues, and bacterial enzymes having approximately 190 residues.

“Concentration” means the activity level of proteases per kg dry matter of a feed composition comprising a feed material treated with the proteases.

“Stable” means that the protease remains active and the feed material does not become moldy, rot, or otherwise deteriorate for at least about one year after treatment.

“Feed composition” means the complex formed by adding enzymes to feed material

“Feed-grade” means non-toxic when fed to animals.

### BRIEF DESCRIPTION OF THE DRAWINGS

FIG. 1 is a graph plotting fermenter pH as a function of hours post-feeding to illustrate the diurnal fluctuation of pH in continuous culture fermenters after feed addition (0900 h) as affected by the enzyme mixture. Values are Least Square Means and vertical bars indicate SEM.

### DETAILED DESCRIPTION OF THE INVENTION

The present invention broadly provides a method of increasing fiber digestion in ruminants by providing a feed additive or feed composition comprising at least one protease. Specifically, the invention providers a method of increasing digestibility of a forage or a grain feed comprising the steps of providing at least one protease; providing a forage or a grain feed suitable for a ruminant animal; applying the protease to the forage or the grain feed to form a feed composition; and administering the composition to the animal, whereby an increase in digestibility is effected.

In another aspect, the invention provides a method of feeding a ruminant animal comprising the steps of providing at least one protease; providing a forage or a grain feed; applying the protease to the forage or the grain feed to form a feed composition; and administering the composition to the animal, whereby an increase in digestibility is effected.

In another aspect, the invention provides a method of treating a forage or a grain feed to increase digestibility comprising the steps of providing at least one protease; providing a forage or a grain feed suitable for a ruminant animal; applying the protease to the forage or the rain feed to form a feed composition; and administering the composition to the animal, whereby an increase in digestibility is effected.

In another aspect, the invention provides a method of producing a feed additive comprising the steps of providing at least one protease; mixing the protease with one or more inert or active ingredients to form the feed additive; and feeding the feed additive to a ruminant animal or adding the feed additive to a forage or a grain feed for the animal, whereby an increase in digestibility is effected.

In another aspect, the invention provides a method of producing a feed composition for feeding to a ruminant animal comprising the steps of providing at least one protease; providing a forage or a grain feed; and applying the protease to the forage or the grain feed to form the composition, whereby an increase in digestibility is effected.

In another aspect, the invention provides a feed additive comprising at least one protease in combination with one or more inert or active ingredients. In another aspect, the invention provides a feed composition for feeding to a ruminant animal comprising a forage or a grain feed in combination with at least one protease, whereby an increase in digestibility is effected.

In another aspect, the invention provides use of a protease for feeding a ruminant animal comprising the steps of providing at least one protease; providing a forage or a grain feed; applying the protease to the forage or the grain feed to form a feed composition; and administering the composition to the animal, whereby an increase in digestibility is effected.

In another aspect, the invention provides use of a protease for producing a feed additive comprising the steps of providing at least one protease: mixing the protease with one or more inert or active ingredients to form the feed additive; and feeding the feed additive to a ruminant animal or adding the feed additive to a forage or a grain feed for the animal, whereby an increase in digestibility is effected.

In yet another aspect, the invention provides use of a protease to produce a feed composition comprising the steps of providing at least one protease; providing a forage or a grain feed; and applying the protease to the forage or the grain feed to form the composition, whereby an increase in digestibility is effected.

Ruminant animals include, but are not limited to, cattle, sheep, goats, camels, buffalo, deer, reindeer, caribou and elk. The forage or grain feed includes, but is not limited to, alfalfa hay and silage, grass hay and silage, mixed hay and silage, straw, corn silage, corn grain, barley silage, barley grain, oilseeds or a combination thereof. Preferred forage includes, but is not limited to, alfalfa and alfalfa mixtures, including alfalfa-grass mixed forages and diets containing alfalfa. The forage or grain feed can be dry (moisture content greater than 15%) or wet (moisture content less than 15%).

The feed additive or feed composition includes proteases as the major component, such that no other enzyme activity is required although other activities may be present. The proteases can include, but are not limited to, cysteine proteases, metalloproteases, aspartate proteases, and serine proteases which maybe trypsin-like or subtilisin-like. It is readily understood by those skilled in the art that proteases can be prepared by several different methods. For example, proteases can be obtained by constructing a host organism to produce desired proteases in particular amounts by standard techniques. Alternatively, proteases can be derived from microorganisms or ferments of microorganisms which contain or are capable of producing such proteases. For example, proteases can be derived from bacteria such as species from the genus Bacillus or from fungi such as species from the genus Trichoderma. Alternatively, commercially available proteases may be used, including but not limited to, the following: Protex 6L(Genencor International, Rochester, N.Y.). Suitable serine proteases include, but are not limited to, the following: alkaline serine endopeptidases with subtilisin-like properties (E.C. 3.4.21.62). Suitable subtilisins include, but a not limited to, the following: Subtilisin Carlsberg (Type VIII, Cat. No. P5380) obtained from Sigma Chemicals, St. Louis, Mo.

The proteases are provided in quantities sufficient to provide a particular concentration and activity to maximize feed digestibility and animal performance. The proteases are applied to the forage or grain feed preferably in an amount in the range of 0.1 to 20.0 mL/kg of dietary dry matter consumed, more preferably in the range of 0.5 to 2.5 mL/kg of dietary dry matter consumed, and most preferably 0.75 to 1.5 mL/kg of dietary dry matter consumed.

The amount of proteases added to the forage or grain feed is such that the resulting forage or grain feed comprises sufficient protease activity in the range of 1,000 to 23,000 protease units/kg dry matter, more preferably in the range of 2,300 to 11,000 protease units/kg dry matter, and most preferably 3,300 to 6,800 protease units/kg dry matter. Protease activity refers to the capacity of the proteases to cleave peptide bonds, or protease activity as assayed at pH 6.0, 39° C. using 0.4% azocasein as substrate.

While subtilisin-like proteases are alkaline (i.e., optimally active above pH 7), suitable proteases preferably exhibit activity in a pH range between 5-7 which corresponds to the pH range characteristic of the rumen.

The invention extends to particular ruminant feed additives and feed compositions. Various formulations of proteases are ideal for administration to ruminants to promote fiber digestion. Proteases can be formulated as a solid, liquid, suspension, feed additive, admixture, or feed composition as follows.

i) Solids—Proteases can be formulated as a solid, as a mineral block, salt, granule, pill, pellet or powder. In the form of a powder, proteases may be sprinkled into feed bunks or mixed with a ration.

ii) Liquids and Suspensions—Proteases can be incorporated into liquids, formulated as solutions or suspensions, by adding lyophilized or powdered proteases to a suitable liquid. Proteases can be mixed with the animal's drinking water or provided in other liquid forms for consumption.

iii) Feed Additive—Proteases can be administered in the form of a feed additive comprising a preparation of lyophilized microorganisms to which proteases are added. The feed additive may be included with the animals' regular feed. A feed additive may comprise at least one feed-grade protease containing 100 to 500,000 units of protease per mL or gram in combination with one or more inert or active ingredients.

iv) Admixture—Incorporation of active ingredients into feed material is commonly achieved by preparing a premix of the active ingredient, mixing the premix with vitamins and minerals, and then adding the premix or feed additive to the feed. Proteases can be admixed with other active ingredients known to those in the art, for example other enzymes including but not limited to cellulases, xylanases, glucanases, amylases, esterases: antibiotics; prebiotics and probiotics. The active ingredients, including proteases alone or in combination with other active ingredients, can be combined with nutrients to provide a premixed supplement. Nutrients include both micronutrients, such as vitamins, minerals, and macronutrients. The premix may then be added to feed materials.

v) Feed Composition—Proteases can be provided in the form of a feed composition comprising a forage or grain feed treated with proteases. Proteases may be mixed with a forage or grain feed in dry form; e.g. as a powder, or as a liquid to be used as a drench or spray for example.

These formulations may be stabilized through the addition of other proteins or chemical agents. Pharmaceutically acceptable carriers, diluents, and excipients may also be incorporated into the formulations. To ensure that the animals consume a sufficient quantity, flavorings may be added to provide proteases in a form which appears palatable to the animal.

Proteases may be administered in several ways; however, oral administration in the animal's feed is preferred. The dosage of proteases depends upon many factors that are well known to those skilled in the art, for example, the type, age, and weight of the animal. The proteases can be administered to the animal on a daily basis.

To achieve the improvement in digestibility of the feed materials, the proteases should be applied to the forage or grain feed in accordance with certain procedures and parameters. With reference to the mass of the forage or grain feed, sufficient powdered or liquid proteases are diluted in water to provide the desired activity level in the range of 1,000 to 23,000 protease units/kg dry matter, more preferably in the range of 2,300 to 11,000 protease units/kg dry matter, and most preferably 3,300 to 6,800 protease units/kg dry matter.

The proteases, such as those in liquid form, are applied to the forage or grain feed to provide an even distribution of the aqueous solution over the forage or grain feed. Typically, the proteases will be sprayed onto the forage or grain feed while the forage or grain feed is simultaneously mixed to encourage an even distribution of the proteases.

Treatment of the forage or grain feed may be combined with various typical feed processing steps which may occur before or after protease treatment. Such processing steps include, without limitation, dry rolling, steam-rolling, steam-flaking, cubing, tempering, popping, roasting, cooking or exploding the feed. When the processing steps include high temperatures, the proteases are preferably applied after processing.

The inventors determined the surprising effectiveness of proteases to increase digestibility of forage or grain feed in ruminants as described in the Examples. As shown in Example 1, twenty-two commercially available enzyme mixtures were initially screened to assess their protein concentration, enzymic activities, and hydrolytic capacity on natural substrates (i.e., reducing sugars released).

Example 2 sets out three experiments involving in vitro ruminal degradation of forages commonly used in ruminant diets. Importantly, the enzyme mixtures were investigated in the presence of ruminal fluid. In Experiment 1, candidate enzyme mixtures were identified and further evaluated in Experiment 2 for their degradative effects on alfalfa and corn silage. Correlations were then performed to establish relationships between these factors. Two enzyme mixtures were thereby selected, and their effects on rate and extent of in vitro forage degradation were further determined in Experiment 3

As shown in Example 3, the effects of a selected protease enzyme mixture on a total mixed ration (used fresh instead of oven- or freeze-dried) was examined using continuous culture. Ruminal metabolic responses can be simulated in vitro by using a dual flow continuous culture fermenter. This system consists of a series of fermenters which are inoculated with ruminal fluid obtained from ruminally-fistulated cattle; continuously fed with the control or test feed material; and continuously infused with artificial saliva. The fermenters maintain temperature, pH, anaerobic conditions and continuous flow of digesta at rates matching those found in ruminants consuming similar diets. Further, the pH was adjusted to yield two different pH ranges (5.4-6.0, and 6.0-6.7) to simulate the reductions in salivation that typically occur when cattle are fed high concentrate diets (Van Soest, 1994). It was investigated whether the protease enzyme mixture would improve the degradability of the diets, and whether the extent of the improvement would be lower at low pH than at high pH. Analyses including bacterial counts, enzymic activities and chemical tests were conducted. Addition of the protease enzyme mixture under different pH conditions enhanced fiber degradation with only a numerical increase in protein degradation. Overall, these findings further suggest that the mode of action of protease enzyme mixtures in ruminants is a combination of direct and indirect effects, exerted both over the feeds and the microbial populations in the rumen.

In Example 4, analysis of a selected protease enzyme mixture further suggested that the type of protease appears to be subtilisin-like, but the beneficial effects on fiber digestion may not be limited to just this type of protease.

Specifically, the inventors have discovered that adding specific protease enzyme mixtures to feeds commonly used in ruminant diets increases fiber (NDF) digestion in the rumen by up to 60% (expected range: 10 to 45%). Furthermore, this increase in fiber digestion is not accompanied by a large, undesirable increase in ruminal protein digestion or by an increase in methane production. The increases in fiber digestion due to added proteases are greatest for alfalfa forage and diets containing some alfalfa forage, but improvements are not limited to alfalfa-based diets.

An increase in fiber digestion of this magnitude is expected to result in an increase in the amount of energy available to the animal, thereby improving growth rate or milk production. The mechanism whereby these proteases increase fiber digestion appears to be related to the removal of proteinaceous entities that serve as structural barriers to fibrolytic microbes and their enzymes. In alfalfa, it seems that effective enzymes work by removing structural barriers that retard the microbial colonization of digestible fractions, increasing the rate of degradation. In corn silage, effective enzymes appear to interact with ruminal enzymes to degrade the forage more rapidly. Given the magnitude of the increases in fiber digestion observed, it is expected that addition of proteases to ruminant diets will improve growth rate or milk production of animals offered these diets.

Example 5 shows that adding the protease enzyme to the diet of dairy cows increased the digestibility of the diet. Digestibility of DM, OM, N, ADF, and NDF were consistently increased due to protease enzyme. The improvement in digestibility was generally greater for a lower forage diet (i.e., a diet typical of that fed commercially to high producing dairy cows) than for a high forage diet; however, the improvements in digestibility were substantial for both diets.

Example 6 shows the increase in digestibility of the individual forages used in the feeding study reported in Example 5. When the individual forage components of the diet were treated separately, protease enzyme improved digestion of alfalfa hay, but not barley silage. However, when these same forages comprised the diet fed to the cows in Example 6, the digestibility of the total diet was increased. The increase in digestibility was greater than what could be explained by just an improvement in digestibility of the alfafa hay component, because the alfalfa hay only comprised 16% of the diet. The increased enzyme activities of ruminal fluid shown in Example 5 indicate that feeding a protease enzyme increased the overall fibrolytic capacity of the rumen, indicating a synergy between the exogenous enzyme action and the ruminal microorganisms. Thus, by adding protease to the diet, the capacity of the rumen to digest fiber was increased. The increase in digestion observed in Example 5 was not limited to just the alfalfa hay component of the diet, as was the case in Example 6 when the forages were incubated separately.

It will be apparent to those of ordinary skill in the art that alternative methods reagents, procedures and techniques other than those specifically detailed herein can be employed or readily adapted to practice this invention. The invention is further illustrated in the following non-limiting Examples. All abbreviations used herein are standard abbreviations used in the art. Specific procedures not described in detail in the Examples are well-known in the art.

### EXAMPLE 1

### Initial Screening of Enzyme Mixtures

Twenty-two commercially available enzyme mixtures were used. Experimental codes (RT1180 to RT1201) were allocated to each enzyme mixture (RT1180 to RT1194 from Genecor Int., Rochester, N.Y.; RT1195 to RT1198 from Quest Int., Naarden, the Netherlands; RT1199 to RT1201 from DSM, Delft, the Netherlands). In addition, three commercial enzyme mixtures of known efficacy served as positive controls; experimental codes P. PD, and PB (Cargill Inc., St Louis, Mo.).

a. Protein Concentration

The amount of protein was determined using the Bio-Rad DC protein determination kit (Bio-Rad Laboratories, Hercules, Calif.) with bovine serum albumin as standard. Five (5) μL of each diluted enzyme mixture was added to microtitre plates, followed by 25 μL of Bio-Rad reagent A and 200 μL of reagent B. The reaction was allowed to proceed for 15 minutes at room temperature, and absorbance was read at 630 nm using a MRX-HD plate leader (Dynatech Laboratories Inc., Chantilly, Va.).

b. Enzymic Activities

i. Polysaccharidase Activity

Polysaccharidase activity was determined in triplicate using substrate solutions or suspensions (1% w/v) in distilled water. Xylan (from birchwood or from oat spelts), carboxymethylcellulose (CMC, medium viscosity), Sigmacell 50, lichenan, laminarin, and soluble starch (all obtained from Sigma Chemicals, St Louis, Mo.) were used for determination of xylanase (EC 3.2.1.8), endoglucanase (EC 3.2.1.4), exoglucanase (EC 3.2.1.91), α-1,3-α-1,4-glucanase (EC 3.2.1.73), α-1.3-glucanase (EC 3.2.1.6), and α-amylase (EC 3.2.1.1), respectively. In addition, barley α-glucan, xyloglucan (from tamarind seeds) and wheat arabinoxylan were obtained from Megazyme International Ltd. (Wicklow, Ireland).

Suitably diluted enzyme (50 μL) and substrate solutions (450 μL) were incubated for 5-60 minutes depending on the activity, and assayed according to Wood and Bhat (1988). Briefly, the reaction was terminated by adding two volumes of Somogyi-Nelson's reagent (Somogyi, 1952), and boiling for 10 minutes. Reducing sugars were determined calorimetrically at 630 nm. One unit of activity was defined as the amount of enzyme required to release 1 μmol equivalent xylose or glucose min⁻¹ g⁻¹ enzyme product, under these assay conditions.

ii. Glycosidase Activity

Glycosidase activities measured were β-D-glucosidase (EC 3.2.1.21), β-D-xylosidase (EC 3.2.1.37), α-L-arabinofuranosidase (EC 3.2.1.55), β-D-galactosidase (EC 3.2.1.23) and acetyl esterase (EC 3.1.1.6) using 1 mM solutions of p-nitrophenyl derivatives (Sigma Chemicals, St Louis, Mo.) as described in Wood and Bhat (1988). One-hundred (100) μL of each substrate was incubated (n=6) with each diluted enzyme mixture (12.5 μL) and buffer (37.5 μL) at 39° C. for 30 minutes, except for acetyl esterase activity. Upon incubation, the reaction was terminated by addition of 150 μL of 0.4M glycine-NaOH buffer (pH 10.8) and the absorbance was measured at 420 nm. For acetyl esterase determination, sequential readings were taken at 0, 5, 10, and 15 minutes of incubation and activity was calculated based on the increase in absorbance at 340 nm. One unit of activity was defined as the amount of enzyme required to release 1 μmol nitrophenol min⁻¹ g⁻¹ enzyme mixture.

iii. Protease Activity

Protease activity was determined using a radial diffusion assay method (Brown, et al., 2001). Ten (10) mL of a 1% (w/v) molter agar (Fermtech Agar, EM Science, Gibbstown, N.J.) prepared in citrate-phosphate buffer (0.1 M, pH 6.0) and containing 0.5% (w/v) gelatin as substrate (Fisher Scientific, Fair Lawn. N.J.) was poured into pet dishes (90 mm diameter), 0.01% sodium azide (w/v) was included to prevent microbial growth. Upon agar solidification, a 6 mm well was made in each plate using a cork borer, and 5 μL of undiluted enzyme mixture plus 20 μL of distilled water were added. The plates were incubated at 39° C. for 16 hours. At the end of the incubation period, the unhydrolyzed gelatin was precipitated by addition of a saturated ammonium sulfate solution. The clear radial areas around the wells (denoting areas degraded by the enzymes) were measured by two independent observers using an electronic digital caliper (Traceable, Model No 62379-531, Control Company, Friendswood, Tex.). The protease activity was then expressed in terms of mm of gelatin degraded, after correction by the well's diameter.

c. Release of Reducing Sugars From Natural Substrates

The hydrolytic potential was determined in triplicate by measuring the reducing sugars released from 25 mg of alfalfa hay or corn silage (freeze-dried and milled to pass a 1 mm screen) after a 15-min incubation at 39° C. and pH 6.0(450 μL of 0.1 M citrate-phosphate buffer) with enzyme mixture (50 μL). Powdered enzyme mixtures were diluted 250-fold with distilled water, whereas liquid enzyme mixtures were diluted 25-fold. Prior to freeze-drying, the substrates were washed with distilled water for 2 hours at room temperature to extract soluble components. Blanks containing substrates only were included for correction. The reducing sugars released were expressed in μg glucose equivalents/mg enzyme product added.

Table 1 shows the protein contents, enzymic activities and reducing sugars released from the incubation of alfalfa hay and corn silage for all enzyme mixtures. The protein content varied among all enzyme mixtures likely due to the diversity of microbial sources, production procedures, and preservatives or carriers commonly used in their formulation. With regard to enzymic activities, RT1197 was the most concentrated of those tested, ranking within the first five preparations in 14 out of the 17 activities determined. RT1191, RT1192, RT1196 and RT1200 also showed high activities in general. RT1191, RT1192 and RT1197 were the most active against cellulose. RT1190, RT1191, and RT1192 were the most successful in releasing reducing sugars from both substrates.

The relationship between enzymic activities and release of reducing sugars from alfalfa hay and corn silage was determined (Table 2). A stepwise regression of protein contents and enzyme activities on the release of reducing sugars showed that protein content alone explained 60% and 59% (P<0.001) of the total variation for alfalfa hay and corn silage, respectively. Activity against α-glucan explained a further 24% (P<0.001) of the variation in alfalfa hay, but its relationship with the release of reducing sugars was negative. In contrast, release of reducing sugars from corn silage was positively correlated to activity against oat spelt xylan (P<0.03), CMC (P<0.07) and crystalline cellulose (P<0.05), but negatively correlated to activity against birchwood xylan (P<0.01), starch (P<0.00(1) and pNP-glucopyranoside (P<0.003). Together, all these variables explained 96% of the total variation in the release of reducing sugars from corn silage. The strong positive relationship between protein content and release of reducing sugars from both substrates may suggest that concentrated enzymes worked better, or at least faster, than more diluted samples, supplying enough enzyme activity to break down polysaccharides to simpler molecules in the short time allocated.

### EXAMPLE 2

### In vitro Rumen Degradation Assessment for Enzyme Mixtures with Protease Activity

Several experiments were carried out to identify enzyme mixtures with superior protease activity in the presence of ruminal fluid, and their effects on alfalfa hay or corn silage. The same batch of feed material was used for all experiments. One (1) g DM of alfalfa hay or corn silage (±20 mg, dried and milled to pass a 2-mm screen) was weighed into 125 mL fermentation bottles (Wheaton Scientific, Millville, N.J). The alfalfa hay contained 382.0 and 252.4 g/kg DM of NDF and ADF, respectively, whereas the corn silage contained 467.4 and 254.1 g/kg DM of NDF and ADF, respectively.

With regard to the statistical analyses, Experiment 1 was a completely randomized design, with a model that included enzyme treatment and substrate as fixed effects. As a significant enzyme-substrate interaction was found, analyses were carried out separately for each forage source (alfalfa hay and corn silage). Differences among means were analyzed using the Mixed Procedures of SAS (SAS Inst. Inc., Cary, N.C., 1996), with the PDIFF command invoked. Protein contents, total activities, and reducing sugars released were correlated to dry matter digestibility (DMD) values for each forage source using the Stepwise Regression Procedures of SAS. Data from Experiments 2 and 3 were analyzed as a completely randomized design with a factorial arrangement of treatments, using a model that included enzyme as fixed effect, and experimental run as a random effect. Unless stated otherwise, significance was declared at P<0.05, whereas trends were discussed at P<0.10.

i. Experiment 1—Effects of Addition of Enzyme Mixtures on Degradation of Alfalfa Hay or Corn Silage

The 22 enzyme mixtures were applied at a rate of 1.5 mg/g DM forage, 20 hours prior to inoculation with ruminal fluid. Three commercial enzyme mixtures were used as positive controls: P, PD, and PB. One-hundred and twenty-five (125) mg of each enzyme mixture were dissolved in 50 mL of distilled water, and 0.6 mL was added to each bottle. Treatments were weighed in triplicate. After 3 hours, 40 mL of anaerobic buffer medium (Goering and Van Soest, 1970) adjusted to pH 6.0 using 1 M trans-aconitic acid (Sigma Chemicals, St Louis, Mo.), was added, and bottles were stored at 25° C. overnight. Ruminal fluid was collected from 3 lactating, ruminally-fistulated dairy cows fed a corn silage-based total mixed ration. Feed was withdrawn from the feeders 4 hours prior to the fluid being collected. Ruminal contents were strained through 4 layers of cheesecloth under a continuous stream of CO₂, and transferred to the laboratory in pre-warmed Thermos flasks. 10 mL of ruminal fluid were inoculated into each bottle already pre-warmed to 39° C. Controls containing substrate only, or ruminal fluid only, were also included in triplicate. Bottles were incubated at 39° C. for 18 hours, and undegraded residues were immediately filtered through pre-weighed sintered glass crucibles (Porosity 1, 100-160 μm pore size). Residues were dried at 110° C. for 24 h to determine apparent dry matter degradation (DMD) expressed as g/kg. The ranking of enzyme mixtures was determined based on their relative increase in DMD with respect to the controls.

Table 3 shows the effects of the enzyme mixtures on alfalfa hay or corn silage. For alfalfa hay, five enzyme mixtures increased (P<0.05) DMD with respect to the untreated controls, after 18 hours of incubation with ruminal fluid. For corn silage, 11 enzyme mixtures increased (P<0.05) DMD. Interestingly, the most effective enzyme mixtures against alfalfa hay were not as effective against corn silage, suggesting a strong enzyme-feed specificity.

The relationship between enzymic activities and the apparent DMD of alfalfa hay and corn silage after 18 hours on incubation with ruminal fluid was examined (Table 4). When a stepwise multiple regression of protein concentrations, total enzyme activities, and reducing sugars release with in vitro rumen degradation values was performed, a positive correlation (P=0.01) between xylanase (oat spelt) and alfalfa DMD was observed. Protease activity was also positively related with alfalfa DMD (P<0.10). However, the proportion of the variance explained by the model was less than 40%. Activity against oat spelt xylan was also significant for corn silage (P=0.04) but the nature of the relationship was negative (Table 4). It is unclear, however, whether this negative correlation indicates a cause and effect relationship between low xylanase activity and high DMD in corn silage.

ii. Experiment 2—Dry Matter Degradation Kinetics of Alfalfa Hay or Corn Silage Treated or Untreated with Selected Protease Enzyme Mixtures

Based upon results for Experiment 1, RT1184 and RT1197 were selected for further evaluation using alfalfa, while RT1181 and RT1183 were selected for studies with corn silage. The Daisy II in vitro fermentation system (ANKOM Corp., Fairport, N.Y.) was used to examine the rate and extent of DM and fiber degradation of forages treated with these enzyme mixtures. Five hundred (500) mg (±20 mg) of alfalfa hay or corn silage were weighed into artificial fiber bags (#F57, ANKOM Corp.) which were then heat-sealed. Groups of 30 bags, including 6 empty bags for correction, were placed upright in plastic containers, together with 150 mL of buffer (pH 6.0). The buffer used for this pre-treatment was according to Goering and Van Soest (1970) without addition of reducing solution. Enzymes were added to the containers at the appropriate rates (1.5 mL/g forage DM), dissolved in 1 mL of distilled water, 20 hours prior to addition of ruminal fluid. The mixtures were gently shaken to allow proper mixing and stored at room temperature (24° C.). Ruminal fluid was collected from three cows as described in Experiment 1.

Four hundred (400) mL of ruminal fluid were then added to each ANKOM fermentation jar, together with 1,600 mL of anaerobic buffer (adjusted to pH 6.0). Bags, plus all liquid contents in the plastic containers, were added to the fermentation jars, and fermentation allowed to continue at 39° C. for 96 hours. Bags were removed in quadruplicate (plus one empty bag per time point) at 0, 6, 18, 30, 48, and 96 hours of incubation, and washed under cold tap water until excess water ran clear. Bags were dried at 55° C. for 48 hours, and DMD was determined. Fiber (NDF and ADF) degradation was determined sequentially on the same bags using the ANKOM²⁰⁰ fiber analysis system (ANKOM Corp., Fairport, N.Y.) according to Van Soest et al. (1991). For the NDF analysis, α-amylase was included but sodium sulfite was excluded. After each analysis, bags were dried as described for DMD determination. The experiment was replicated twice.

Table 5 shows the dry matter degradation kinetics of alfalfa hay or corn silage treated or untreated with the enzyme mixtures. RT1184 increased (P<0.05) the degradation of alfalfa hay after 6 hours (+9.0%), with a trend (P<0.10) towards improving the degradation at 0 hours (+8.8%). No differences were detected after 6 hours of incubation for any of the treatments in alfalfa. In corn silage, RT1181 increased (P<0.05) DMD after 6 hours of incubation, and tended to increase (P<0.10) DMD at 30 hours. In addition, RT1181 and RT1183 increased (P<0.05) DMD at 48 hours. The latter is surprising given the general agreement that enzymes increase the rate, but not extent, of degradation (Colombatto, 2000; Beauchemin et al., 2001). However, DMD at 48 hours was not an end-point for corn silage, as considerable degradation still took place after this time (between 10 and 14 percentage units). It is likely that active degradation was still under way during the 30-48 hour incubation period, in contrast to what was observed in alfalfa hay.

Table 6 shows the fiber (NDF, ADF, and hemicellulose) degradation kinetics for alfalfa hay. RT1184 increased (P<0.05) the hemicellulose degradation of the alfalfa hay at 6 hours of incubation, almost by 100%, whereas sizeable increases (albeit non-significant) were observed in NDF after 6 and 18 hours of incubation for the same enzyme treatment. In contrast, RT1197 failed to show differences with respect to the control. It is evident that most of the available fiber had been degraded by 48 hours, and that enzymes merely increased the rate of degradation. The fact that very little of the fiber fraction was degraded at 0 hours, coupled with the increased hemicellulose degradation after 6 hours, strongly suggests that RT1184 removed some components that presented a physical barrier to degradation. The fact that RT1184 contains mainly protease activity may suggest that protein is the component being removed.

Table 7 shows the fiber (NDF, ADF and hemicellulose) degradation kinetics for corn silage. RT1181 increased NDF and ADF degradation at all times up to 48 hours incubation, the values achieving significance (P<0.05) at 18 and 48 hours. Hemicellulose degradation was increased (P<0.05) by the same enzyme at 6 hours incubation, and tended to be higher (P<0.10) than the controls at 18 hours (+17%) and 48 hours (11%). In contrast to alfalfa hay, there was no indication of “pre-ingestive” effects (i.e., 0 hour differences) between the controls and any of the enzyme treatments. This finding suggests that, with corn silage, the enzyme mixtures worked only at the ruminal level. Alfalfa appears to benefit by a pre-treatment period, possibly due to small structural changes to the cell wall (Nsereko et al., 2000), whereas the situation in corn silage is unclear. It thus appears that the optimal length of an enzyme-feed interaction time prior to feeding may depend on the type of forage.

Table 8 shows the degradation profiles of the non-fiber fractions to determine the proportion of the increase in DMD attributable to the fiber fraction. When RT1184 was added to alfalfa, fiber degradation explained about a third of the DMD during the first 18 hours incubation. When RT1181 was added to corn silage, fiber degradation contributed to at least 50% of the total increase in degradation, with the significant increases in DMD found at 48 hours being almost totally explained (86.4%) by an increase in fiber degradation. These findings further confirm that RT1181 and RT1184 have different modes of action. It seems that RT1181, which is derived from Trichoderma longibruchiatum, concentrates its action on the fiber once in the in vitro rumen system. RT1184, which is derived from Bacillus spp., acts mainly on the non-fibrous fraction (possibly protein), with the effects evident at the 0 hours incubation, suggesting the removal of structural barriers that retard microbial colonization and degradation or alfalfa.

iii Experiment 3—Effects of Selected Protease Enzyme Mixtures in Combination or on Mixed Forage

Since Experiment 2 indicated that RT1181 and RT1184 effectively degraded corn silage and alfalfa hay respectively, the inventors examined whether these enzymic mixtures would be effective on a mixed forage (1:1, w/w of alfalfa hay and corn silage) or when the enzymic mixtures were combined (“8184”). The method was identical to that described in Experiment 2. The treatment groups were as follows:

1. control (no enzyme)

2. RT1181 alone

3. RT1184 alone

4. combination of RT1181 and RT1184 (1:1, v/v) at two final levels, 0.5 (8184 Low) or 1.5(8184 High) mL/g forage DM.

As shown in Table 9, RT1184 increased (P<0.05) DMD of the alfalfa-corn silage combination at 6 and 18 hours incubation. It also increased (P<0.05) DMD at 0 hours, indicating the presence of “pre-ingestive” effects. Moreover, the degree of improvement with respect to the controls remained fairly constant between 0 and 18 hours, which suggests that the improvement at 0 hour was not achieved at the expense of the most readily digestible fractions (i.e., those degraded within the first 12 hours incubation). That would have been the case had the degradability at 6 or 18 hours been equal to that of the controls. Available evidence suggests that degradation rate started to slow down between 18 and 30 hours incubation, consistent with the time at which fiber fractions are attacked by ruminal microbe when incubated in vitro.

Analysis of the fiber degradation in the RT1184 treatment indicated that the increase in DMD was accompanied by an increase (P<0.05) in NDF degradation at 6 hours and a trend (P<0.10) towards an increase in NDF degradation at 18 hours, and an increase in hemicellulose degradation at 6 and 18 hours (Table 10).

The combination of RT1181 and RT1184 showed intermediate values between the controls and RT1184 (Table 9), and treatment 8184 High tended (P<0.10) to increase DMD at 6 hours incubation, accompanied by an increase (P<0.05) in NDF and hemicellulose degradation. As RT1181 failed to significantly increase DMD or fiber degradation, it is reasonable to speculate that all increases found in the alfalfa corn combination were due to the action of RT1184 alone. Furthermore, it seems that RT1184 application rate could be halved without losing effectiveness in fiber degradation.

Of particular interest was the fact that RT1184 and the two combinations of RT1181 and RT1184 increased (P<0.05) both DMD and NDF end-point (96 hours) degradation. This is in contrast with what is generally observed when enzymes are added to forage (Yang er al., 1999; Colombatto, 2000). Although the increases in DMD are unlikely to be of biological significance, the extent of the improvement achieved with NDF degradation (+2.0, +3.5, and +3.5% for RT1184, 8184 Low, and 8184 High, respectively) is encouraging, especially when the treatments including RT1184 and 8184 High showed higher NDF degradation values at almost all incubation times.

When degradation profiles of the non-fiber fractions were considered, it was found that the increases observed with RT1184 during the first 18 hours incubation could not be attributed only to an increase in the fiber fraction, as the latter fraction explained between 25 and 50% of the increase in DMD. These findings concur with those of Experiment 2, indicating that RT1184 acts mainly on non-fiber fractions, and was effective on mixed forage as well as pure alfalfa hay alone.

### EXAMPLE 3

### Effects of a Selected Protease Enzyme Mixture on Enzymic Activity, Microbial Numbers and Fiber Degradation of Total Mixed Ration

The effects of a selected protease enzyme mixture on a total mixed ration were examined. Further, two fermentation pH ranges (5.4-6.0, and 6.0-6.7) were maintained by adjusting the concentration of the artificial saliva. It was investigated whether the enzyme mixture would improve the degradability of the diets, and whether the extent of the improvement would be lower at low pH than high pH.

a. Preparation of Feed Material

The total mixed ration (TMR) consisted of 30% alfalfa hay, 30% corn silage and 40% rolled corn grain (DM basis) which is typical of a commercial diet fed to dairy cows in mid to late lactation. The forage:concentrate ratio was thus 60:40. The alfalfa hay was ground to pass a 4.5-1 mm screen (Arthur H. Thomas Co., Philadelphia. PA), while the rolled corn was ground in a Knifetec 1095 sample mill (Foss Tecator, Höganäs, Sweden) for 2 seconds to achieve partial rupture of the grains. Both substrates were stored at room temperature until use. Corn silage was sampled from different sites within a bunker silo located at the Lethbridge Research Centre (Lethbridge, AB) and stored at 40° C. until use. When required, a sample of the silage (enough for 3 days of feeding) was thawed ad ground fresh for 10 seconds using the Knifetec 1095 sample mill (Foss Tecator, Höganäs, Sweden). Ground samples were stored at 4° C. for a maximum of 3 days. The TMR was prepared every three days in 1 L plastic containers by weighing the individual feed components. The contents were mixed thoroughly and stored at 4° C. Table 11 summarizes the chemical composition of the individual feed materials and of the TMR.

b. Enzyme Mixture and Determination of Protease Activity

The commercially available enzyme mixture RT1184 was used in this study. The enzyme mixture is derived from Bacillus licheniformis, and contains negligible amounts of cellulase, hemicellulase and α-amylase activities (Colombatto et al., 2003).

Protease activity was determined at pH 6.0 and 39° C. using 0.4% (wt/vol) azocasein as substrate (Bhat and Wood, 1989). Briefly, a reaction mixture containing 0.5 mL azocasein, 0.5 mL citrate-phosphate buffer, and 25 μL of enzyme (diluted 1:100 in distilled water) was incubated at 39° C. for 15 minutes. The unhydrolyzed azocasein was precipitated by adding 80 μL of 25% (wt/vol) trichloroacetic acid and then removed by centrifugation at 2,040×g, for 10 minutes at room temperature. A 0.5-mL supernatant sample was mixed with 0.5 mL of 0.5 M NaOH and the absorbance read at 420 nm against a reagent blank. Enzyme (no substrate) and substrate (no enzyme) blanks were also included for correction. One unit of protease activity was defined as the absorbance measured at 420 nm by the action of 10 μg of a standard protease (Streptomyces griseus, Type XIV, Sigma Chemicals, St Louis, Mo.), assayed under identical conditions. The protease activity of the enzyme mixture was determined to be 4507 units/mL (SD=161.0, n=5) calculated as follows:

10 μg of standard gave an absorbance of 0.278

25 μL of a 1:100 diluted solution of the enzyme mixture gave an absorbance of 0.313

Thus, if 1 protease unit was 0.278, the solution contained (0.313/0.278) units=1.126 units. To transform this into units per mL, the dilution factor (100) and the amount added (25 μL) are used:

1.126×40×100=4,507 units/mL undiluted enzyme mixture.

C. In Vitro Rumen Degradation Assessment

Three lactating dairy cows were used in the experiment. Cows were cared for in accordance with the guidelines established by the Canadian Council on Animal Care (1993), and were ruminally-fistulated. Cows were fed a similar diet as that provided to the fermenters.

A four-unit dual flow continuous culture system (similar to that described by Hoover, et al. 1989) was used in four consecutive periods. Ruminal fluid inoculum was collected from the animals 2 hours post-feeding. Ruminal contents were homogenized in a Waring blender (Waring Product Division, New Hartford, Conn.) for 1 minute under a stream of oxygen-free CO₂. The homogenate was then strained through four layers of cheesecloth and transferred to the laboratory in pre-warmed Thermos flasks. Anaerobic conditions were maintained by infusion of CO₂ at a rate of 15 mL/min. Artificial saliva was infused continuously into the fermenters (McDougall, 1948). During each period, two fermenters received saliva at the normal concentration, while two other fermenters received saliva diluted in distilled water to obtain a concentration equivalent to 60% of the normal. The artificial saliva contained 0.2 g/L of urea to simulate recycled nitrogen and 0.015 g of ammonia ₁₅N ((₁₅NH₄)₂SO₄, 10.6% atom percentage ₁₅N; Isotec, Miamisburg, Ohio). The daily amount of ₁₅N provided into each fermenter was about 1.5 mg. Liquid and solid dilution rates were kept constant at 10 and 4.5%/h, respectively. A total of 80 g of DM per day was fed in two equal meals at 0900 and 2100 h. The four treatment groups were as follows:

| Treatment Group | | pH range | Artificial Saliva |
|---|---|---|---|
| HC | high pH with control TMR | 6.0-6.6 | normal |
| HT | high pH with TMR treated | 6.0-6.6 | normal |
| | with enzyme mixture | | |
| LC | low pH with control TMR | 5.4-6.0 | diluted (60% of normal) |
| LT | low pH with TMR treated | 5.4-6.0 | diluted (60% of normal) |
| | with enzyme mixture | | |

For application of the enzyme mixture, 60 μL of enzyme mixture was dissolved into 440 μL of distilled water and added to 40 g TMR (DM basis) in 250-mL plastic containers which were mixed by inversion. The control treatments received 500 μL of distilled water. The interaction period of enzyme mixture and feed material ranged between 12 and 24 h at 4° C.

The experimental design was a 4×4 Latin square with four 9-day periods, each consisting or 6 days for adaptation and 3 days for sampling. On sampling days, collection vessels were maintained at 4° C. to impede microbial action. Solid and liquid effluents were mixed. A 250 mL sample was centrifuged at 16,000×g for 40 minutes at 4° C. to determine effluent DM (i.e., the undigested portion). A second 500 mL sample was centrifuged at 16,000×g for 40 minutes at 4° C. to obtain sediments which were dried at 55° C. and analyzed for ash, nitrogen, NDF, ADF, acid detergent lignin (ADL) and starch.

On days 1 and 2 of each sampling period, fermenter pH was measured every hour from 0800 to 2100 h using a pH probe inserted into the fermenters. Fluid samples from the filtrate were obtained immediately before feed provision in the morning, and then at 2 h, 5 h, 8 h, and 12 h after feed provision for ammonia and volatile fatty acid (VFA) determination. A 5 mL sub-sample of filtered fluid was acidified with 1 mL of 1% sulfuric acid (v/v) for ammonia determination. Another 5-mL sub-sample was acidified with 1 mL of 25% metaphosphoric acid (w/v) for VFA analysis. The samples were stored frozen at −40° C. until analysis. Six hours after the morning feed provision (i.e., 1500 h), gas samples were taken for analysis of gas composition (CO₂ and CH₄) Simultaneously, a 2.0 mL sample of ruminal fluid from the fermenters was removed to quantify total and cellulolytic bacteria. An additional 1.5 mL sample was obtained for determination of enzymatic activities.

Bacteria were isolated from the fermenters on the last day of each period. Fermenter contents were homogenized at slow speed for 1 minute using a Waring blender (Waring Products Division. New Hatford, Conn.) to dislodge solid-phase bacteria, and then strained through four layers of cheesecloth. The filtrate was centrifuged at 1.196×g for 15 minutes at 4° C. to remove feed particles and protozoa, and then at 16.000×g for 40 minutes at 4° C. to isolate the bacterial pellet. The pellets were lyophilized, further ground using a mortar and pestle, and then analyzed for ₁₅N enrichment. Apparent and true (i.e., corrected by microbial portion) digestion of DM, OM, and N were calculated. Digestion of NDF, ADF, ADL and starch were also determined.

i. Statistical Analysis

Data were analyzed using the Mixed procedures of SAS (SAS Inst. Inc., Cary, N.C.) using a model which included pH, enzyme and their interaction as fixed effects. Fermenter and period were considered random effects. Differences among means were declared significant at P<0.05, whereas trends were discussed at P<0.15 unless stated otherwise.

ii. Bacterial Counts

To quantify total viable bacteria, anaerobic serial dilutions (10⁻⁶ to 10⁻⁰) of filtered fermenter contents were prepared using a medium containing 0.1% peptone, 0.1% 0.1% resazurin, 0.05% cysteine, and 0.35% Na₂CO₃ (Bryant and Burkey, 1953). Each dilution was inoculated in triplicate into separate roll tubes containing cellobiose, xylan, starch, and glucose (0.5 mg/mL each). Viable colonies were enumerated after 48 hours of incubation at 39° C. Cellulolytic bacteria were enumerated following a 14 day incubation at 39° C. in triplicate tubes with each of the dilutions (10⁻¹ to 10⁻⁴) using Whatman No. 1 filter paper as the sole carbohydrate source. The most probable number procedure was used (Garthright, 1998). Prior to statistical analysis, microbial data were subjected to log transformation to normalize the distribution of the error (Dehority et al., 1989).

iii. Assay of Enzymic Activities,

Enzymic activities in the liquid phase were determined according to Colombatto, et al., 2003. Endoglucanase (EC 3.2.1.4), exoglucanase (EC 3.2.1.91), β-D-glucosidase (EC 3.2.1.21), xylanase (EC 3.2.1.8), β-D-xylosidase (EC 3.2.1.37), protease, and α-L-arabinofuranosidase (EC 3.2.1.55) activities were determined.

Xylanase and Endoglucanase

Oat spelt xylan and medium viscosity carboxymethylcellulose at a concentration of 10 mg/mL (Sigma Chemicals, St Louis, Mo.) were used as substrates for xylanase and endoglucanase, respectively. 40 μL of enzyme were incubated with 1 mL substrate. 0.90 mL buffer (0.1 M citrate-phosphate buffer, pH 6.0), and 0.06 mL distilled water. Incubations were performed in triplicate for 60 minutes (xylanase) or 120 minutes (endoglucanase) at 39° C. Enzymatic reactions were terminated by adding dinitrosalicylic acid reagent and absorbance was read at 530 nm using a MRX-HD plate reader (Dynatech Laboratories Inc., Chantilly, Va.). The absorbance values were converted to reducing sugars using standard xylose or glucose curves developed under identical conditions. Blanks, substrate alone (i.e., no enzyme) and enzyme alone (i.e., no substrate) were also included to correct for substrate autolysis and sugars present in the enzyme sample, respectively. One unit of activity was defined as the amount of enzyme required to release one nmol of xylose or glucose equivalent min⁻¹ under these assay conditions.

Protease Activity

Protease activity was assayed at pH 6.8 using a 0.4%0 (w/v) solution of azocasein as described above, except that incubation time was 120 minutes, and 40 μL of sample were incubated. One unit of protease activity was defined as the absorbance measured at 420 nm by the action of 1 μg of a standard protease (Streptomyces griseus, Type XIV, Sigma Chemicals, St Louis, Mo.) assayed under identical conditions and simultaneously to each incubation series. 1 μg was used as a standard due to the different assay lengths. If 10 μg had been used, the absorbance would have been too high to fall within the linear range of optical density.

Aryl-Glycosidase Activity

Stock solutions (1 mM) of p-nitrophenyl (p-NP) derivatives were used. Substrates were p-NP-β-D-cellobioside, p-NP-β-D-glucopyranoside, p-NP-β-D-xylopyranoside, and p-NP-α-L-arabinofuranoside (Sigma Chemicals, St Louis, Mo.). Undiluted enzyme samples (20 μL) were incubated with 80 μL of corresponding substrate (prepared in buffer pH 6.0) at 39° C. for 180 minutes. The reaction was terminated by addition of one volume of glycine-NaOH buffer (0.4 M, pH 10.8). Release of p-nitrophenol was determined colorimetrically at 420 nm. One unit of enzyme activity was defined as the amount of enzyme required to release one nmol p-nitrophenol min⁻¹ under these assay conditions.

iv. Chemical Analyses

The following chemical analyses were conducted:

| Parameter Analyzed | Method of determination |
|---|---|
| Effluent dry matter (i.e., undigested | Drying at 55° C. in a forced-air oven for 48 hours |
| portion) | |
| Dry matter (DM) content of diets and | Drying at 110° C. for 24 hours |
| bacterial samples | |
| Organic matter (OM) | Difference following ashing at 500° C. overnight |
| Crude protein (CP) (N × 6.25) of | Flash combustion, chromatographic separation, and |
| samples | thermal conductivity (Carlo Erba Instruments, Milan, |
| | Italy) according to AOAC (1990) |
| Neutral (NDF) and acid (ADF) | ANKOM²⁰⁰ fiber analyzer (ANKOM Corp., Fairport, |
| detergent fiber | NY) according to Van Soest, et al. (1991). Heat-stable |
| | amylase was used during the NDF procedure, but |
| | sodium sulfite was omitted. |
| Starch | Enzymatic hydrolysis of α-linked glucose polymers |
| | according to Rode. et al. (1999) |
| Ammonia content | Modification of the Berthelot reaction (Verdouw, 1978) |
| Volatile fatty acids (VFA) | Separated and quantified by gas chromatography |
| | (Hewlett Packard 5890, Agilent Technologies, |
| | Mississauga, ON) using a 30 m (0.32 mm i.d.) fused |
| | silica column (Nukol column, Sigma-Aldrich Canada |
| | Ltd., Oakville, ON) |
| Lactic acid contents at 2 hours post- | Derivatization with boron trifluoride-methanol (14% BF₃ |
| feeding | in methanol) according to Supelco Bulletin No. 856 |
| | (1998) |
| Resultant methyl esters | Gas chromatography using helium as a carrier (28 cm/s). |
| | A sample of methyl DL-lactate was run to |
| | confirm the retention time of the derivative. |
| Gas composition (carbon dioxide and | Headspace samples of gas were removed 6 hours post- |
| methane) | feeding via the port (with an inserted GC septum) into a |
| | 10 mL syringe fitted with a 26 gauge needle (leur-lock). |
| | The sample was immediately injected into an evacuated |
| | 1 dram vial, and analyzed by gas chromatography |
| | (Micro GC CP3900, Varian Specialties Ltd., Brockville, |
| | ON) using a 10-m PoraPlot Q column. |
| Enrichment of ₁₅N in the bacterial | Flash combustion (Model 1500, Carlo Erba Instruments, |
| pellets isolated from the fermenter | Milan, Italy) with isotope ratio mass spectrometry (VG |
| contents | Isotech, Middlewich, UK). A correction for natural |
| | abundance of ₁₅N-enriched bacteria was made by |
| | running an additional experimental period without |
| | infusion of ₁₅N using two fermenters at high pH and two |
| | at low pH. Bacterial production was estimated by the |
| | ratio of ₁₅N flow in the effluent to ₁₅N enrichment of |
| | the bacterial pellet. |

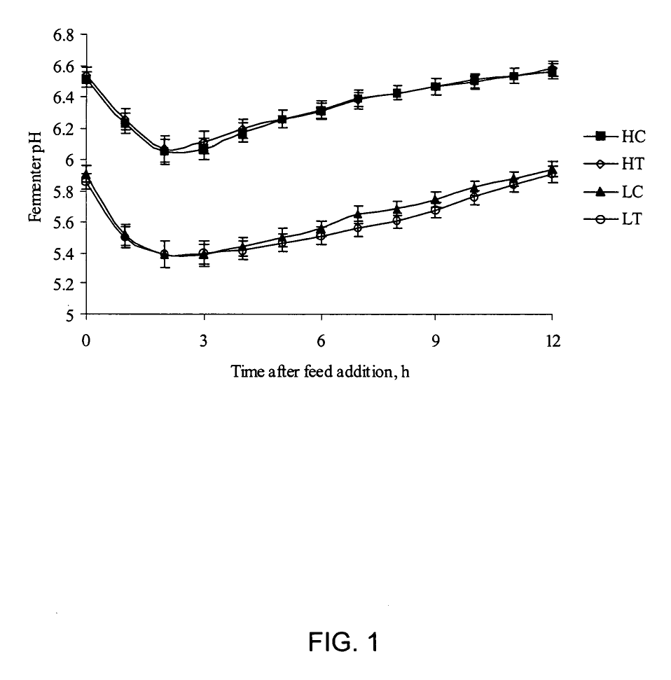

FIG. 1 shows the range of pH obtained by altering the saliva concentration to obtain two different pH profiles. Table 12 shows the effects of pH and enzyme mixture on the total viable bacteria and cellulolytic bacteria. The counts of total viable bacteria increased at low pH (P<0.03) and with addition of the enzyme mixture (P<0.13). Cellulolytic bacteria were reduced at low pH (P<0.02) but remained unaffected by the enzyme mixture (P>0.88).

Table 13 shows the effects of pH and the enzyme mixture at 6 hours post-feeding. Endoglucanase and β-D-xylosidase activities were lower at low pH (P<0.05), whereas exoglucanase activity was reduced (P<0.11). In contrast, protease activity was higher at low pH (P<0.001), largely due to the increase in activity shown by the LT group. The enzyme mixture increased xylanase, endoglucanase, and protease activity (P<0.02), and increased β-D-glucosidase (P<0.07) and exoglucanase (P<0.12). A significant pH x enzyme interaction (P<0.05) was detected in β-D-xylosidase, as the enzyme mixture appeared to increase this activity at high pH, but decrease it at low pH. For protease activity, the significant pH x enzyme interaction was due to the large increase in activity shown by the LT group as previously mentioned. Only α-L-arabinofuranosidase remained unaffected by pH or the enzyme mixture.

Table 14 shows the effects of pH and enzyme mixture on DM, OM, NDF, ADF and starch. True OM digestibility was lower at low pH (P<0.05); however, true DM digestibility only tended to be lower (P<0.07). The enzyme mixture did not affect true DM (P>0.36) or OM (P>0.27) digestibility. NDF and ADF digestion was greatly reduced at low pH (P<0.001), while the enzyme mixture increased NDF digestibility (P<0.005). The enzyme mixture increased hemicellulose digestibility (P<0.001), but did not affect cellulose digestibility. Both true crude protein (CP) and starch degradation were unaffectcd by the treatments (P>0.15).

Table 15 shows the effects of pH and enzyme mixture on VFA production, lactic acid and gas concentrations. Total VFA production was lower at low pH (P<0.006). The branched-chain volatile fatty acids (BCVFA) production also showed a reduction with low pH (P<0.001) High pH increased the proportions of acetate, butyrate, iso-butyrate, and iso-valerate (P<0.01), with caproate showing a trend towards an increase (P<0.14). However, high pH reduced the proportions of propionate and valerate (P<0.01). The acetate:propionate ratio was lower at low pH the at high pH (P<0.001). The enzyme mixture had no effect on any of the VFA (P>0.20). The levels of lactic acid were low and probably not biologically meaningful, however a trend towards higher levels at the high pH was observed (P<0.10). For the total gas composition, the proportion of methane was greatly reduced by low pH (P<0.001), while the CO₂ proportion was higher at high pH (P<0.04).

Table 16 shows the effects of pH and enzyme mixture on nitrogen metabolism of the ruminal microorganisms. Total N flow was higher at high pH (P<0.15), but reduced by the enzyme mixture (P<0.08). Neither bacterial nor dietary N flow was affected by the treatments (P>0.15). The ammonia levels were extremely low, and were higher at high pH (P<0.003) and the enzyme mixture (P<0.07). As a result, the efficiency of microbial protein synthesis tended to be higher at high pH than at low pH (P<0.10).

Addition of the protease enzyme mixture greatly increased fiber (mostly hemicellulose) degradation (up to 43% compared to an untreated control), with numerical increases in dry matter and protein degradation (by 4.5 and 5.5%, respectively). These increases were concurrent with an increase in total microbial numbers and with an increase in the activity of their secreted enzymes. Overall, these findings are consistent with the hypothesis that addition of this protease removes structural barriers present in the forage, allowing a more rapid access to the substrate by the ruminal microbes, which in turn results in faster microbial multiplication and degradation of the substrate. Methane production was decreased at low pH, but was not affected by addition of the protease enzyme mixture. Such results also indicate that the protease enzyme mixture is beneficial in increasing fiber digestibility without increasing methane production by the ruminant which is detrimental to the environment. Further, the effects of the protease enzyme mixture are larger are higher pH conditions which are characteristic of those within the rumen.

### EXAMPLE 4

### Determination of the Type of Protease in the Protease Enzyme Mixture

The protease enzyme mixture (RT1184) of Example 3 was further evaluated to determine the type of promise within the mixture. Protease activity assays were carried out with or without addition of specific protease inhibitors, such as phenylmethylsulfonyl fluoride (PMSF, inhibitor of serine proteases), EDTA (inhibitor of metalloproteases) and p-chloromercuribenzoate (CMB, inhibitor of cysteine proteases). The molecular size of the proteins pent in the mixture was resolved using SDS-PAGE techniques. To determine whether the fraction responsible for the effects was heat-labile, in vitro degradation studies were conducted using both the enzyme both in its, native form (i.e., as is) or after autoclaving (i.e., subjecting the enzyme to 121° C. and high pressure for at least 30 min). Likewise, a dose-response study was carried out to examine the effect of adding incremental enzyme levels on the degradation parameters. Finally, samples from 0 h incubation (i.e., pre-treatment before addition of ruminal fluid) and 18 h of incubation with ruminal fluid were analyzed qualitatively using electron microscopy techniques.

Inhibitor studies showed that only one type of proteases, serine proteases, was present. Addition of 1 mM disodium EDTA or 0.1 mM CMB did not inhibit the proteolytic action, whereas 3 mM PMSF inhibited protease by 36%, thus indicating the presence of serine proteases but absence of metalloproteases in the enzyme mixture. Judged by SDS-PAGE, the enzyme mixture contained a major band of 32 kDa, with other smaller bands of around 22 and 10 kDa.

In vitro rumen degradation assessment demonstrated that, added at 1.5 μL/g DM 2 h prior to ruminal fluid addition, the enzyme mixture was effective at increasing the DM degradation (22 h incubation) of alfalfa hay by 11.8%. Furthermore, degradation was increased up to 21% with increasing application rates (up to 10 μL/g), however the relationship was quadratic (P<0.001, R²=0.85). Autoclaving destroyed this ability, and also eliminated all the positive effects on fiber digestion previously observed with the native (i.e., non-autoclaved) enzyme, indicating that the active component is heat-labile.

Microscopy studies revealed that the enzyme mixture increased the degraded areas of alfalfa hay after 18 h of incubation with ruminal fluid, with some effects also observed at 0 h (i.e., pre-treatment effects). It is speculated that the protease mixture removes structural barriers present in the forages, thus allowing a more rapid colonization and degradation of the fiber by ruminal microorganisms.

These findings suggest that the active principle was heat-labile, most likely the protease activity. An additional in vitro study was conducted using a commercial purified source of serine proteases (Subtilisin, obtained from Sigma Chemicals, St. Louis, Mo.) as a comparison against this enzyme mixture. Application rates were adjusted to provide similar protease activity to that provided by this enzyme mixture. It was shown that purified subtilisin acted in a very similar way to this enzyme mixture, further suggesting a role for this specific type of protege in the observed increases in fiber digestion.

The inventors have thus found that a specific protease with subtilisin-like characteristics increases fiber digestion when added to a range of ruminant feeds. These effects are concurrent with some increases in protein digestion and are believed to stem from the removal of structural barriers (probably proteinaceous in origin) present in the feeds, thereby allowing a more rapid access to the substrates by the ruminal microorganisms. Given the magnitude of the increases in fiber digestion observed, it is expected that addition of proteases to ruminant diets will improve growth rate or milk production of animals offered these diets.

### EXAMPLE 5

### Effects of Addition of a Selected Protease Enzyme Mixture to a Total Mixed Ration on Nutrient Digestibility

The effects of addition of a selected protease enzyme mixture to a total mixed ration (TMR) fed to dairy cows were examined. Further, effects on nutrient digestibility in the total digestive tract were assessed.

a. Animals and Experimental Design

Eight multiparous lactating Holstein cows were used, with four cows surgically fitted with ruminal cannulas. Cows averaged 63±32 (mean±SD) days in milk at the start of the experiment. Average body weight was 690±44 (mean±SD) kg at the beginning of the experiment and 685±40 (mean±SD) kg at the end of the experiment.

The design of the experiment was a double 4×4 Latin square with each period lasting 21 days (10 days of treatment adaptation and 11 days of data collection). Cows were assigned to square by whether they were cannulated and the two squares were conducted simultaneously. During each period, cows received one of four diets. Treatments were arranged as a 2×2 factorial (two levels of forage in the diet, with and without enzyme supplementation).

b. Diets and Preparation of Feed Material

Two diets containing either a high or a low level of forage were used. The high forage diet contained 60% forage, while the low forage diet contained 34% forage (DM basis). Each diet was fed either with or without exogenous protease enzyme to form four treatment groups as follows:

| Treatment Group | Description |
|---|---|
| High Forage, without | High forage control without protease enzyme |
| protease | |
| High forage, with protease | High forage with protease enzyme |
| Low Forage, without | Low forage control without protease enzyme |
| protease | |
| High Forage, with protease | Low forage with protease enzyme |

The forage component of the diet consisted of a mixture of alfalfa hay and barley silage. The concentrate contained steam-rolled barley, dry-rolled corn and a pelleted supplement. The diet was formulated using the Cornell-Penn-Miner System (CPM Dairy, Version 2.0) and was balanced to provide sufficient metabolizable energy and protein, vitamins, and minerals to produce 40 kg/d of milk with 3.5% fat and 3.3% protein. Table 17 shows the chemical composition of the diets.

c. Selected Protease Enzyme Mixture

The enzyme product used in this study was a commercially available protease (Protex 6L ^{Genencor International, Rochester, N.Y.). It was added at a rate of} [symbol] 1.25 Ml/kg of diet DM. This commercial enzyme product is characterized with protease activity derived from a strain of Bacillus licheniformis, compliant with the current specifications for food-grade enzymes and is generally recognized as safe. The enzyme product was sprayed onto the concentrate at the time of manufacturing. The concentrate was then mixed with the forage daily to produce the TMR.

d. Feeding and Management of Animals

Diets were fed as a TMR for ad libitum intake with at least 10% of daily feed refusal. All cows were individually fed three times daily, and had free access to water. Cows were cared for according to the Canadian Council on Animal Care guidelines (Ottawa, ON, Canada). Cows were housed in individual tie stalls fitted with rubber mattresses and bedded with wood shavings and were milked twice daily. Cows were turned outside on a dry-lot for exercise daily.

e. Feed Sampling

Feed offered and refused were measured and recorded daily. Barley silage, chopped alfalfa hay, and concentrates were sampled weekly to determine DM content. Diets were adjusted to account for changes in DM content. Samples of the TMR fed and refused were collected daily, dried at 55° C., ground to pass a 1-mm screen (standard model 4; Arthur H. Thomas Co. Philadelphia, Pa.), and stored for subsequent analyses.

f. Digestibility

Apparent total tract digestion of nutrients was measured using YbCl₃ (Rhône-Poulenc, Inc., Shelton, Conn.) placed directly onto the pelleted concentrate portion of the feed at a rate of 9.7 g YbCl₃/d/cow in order to achieve an intake of 2 g Yb/d/cow. Fecal samples (from the rectum) were collected from all cows from day 6 to 12 at various times during the day. Samples were composited across sampling times for each cow, dried at 55° C., ground to pass a 1-mm screen (standard model 4), and stored for chemical analysis. Apparent total tract nutrient digestibilities were calculated from concentrations of Yb and nutrients in diets fed, orts, and feces using the following equation:

Apparent digestibility=100−(100×(Yb_{d}/Yb_{f})×(N_{f}/N_{d})) (1)

where Yb_{d}=Yb concentration in the diet consumed (i.e., offered orts),

Yb_{f}=Yb concentration in the feces,

N_{f}=concentration of the nutrient in the feces, and

N_{d}=concentration of the nutrient in the diet consumed (i.e., offered orts).

g. Ruminal Sampling

For the determination of enzyme activities, animal contents were sampled from the cannulated cows 0 and 4 hours after the afternoon feeding on days 19 and 20. Approximately 1 L of ruminal contents was obtained from the anterior dorsal, anterior ventral, medial ventral, posterior dorsal, and posterior ventral locations within the rumen, composited by cow, and strained through PeCAP® polyester screen (pore size 355 μm; B & S H Thompson, Ville Mont-Royal, QC, Canada). Residual solids strained from whole ruminal contents were combined (1:1, wt/vol) with 0.9% NaOH, homogenized in a blender (Waring Products Division, New Hartford, Conn.) for 2 min, r-strained through PcCAP® polyester screen (pore size 355 μm), and mixed with the filtered ruminal fluid. Fifty milliliters of the ruminal fluid resulting from the two-step filtering process was sampled. All samples were stored at −20° C. until analysis of enzyme activities.

h. Laboratory Analyses

The following analyses were conducted:

| Analysis | Methodology |
|---|---|
| Feed | |
| Analytical dry matter (DM) | Oven drying at 135° C. for 3 hours |
| Organic matter (OM) | Ashing |
| Nitrogen (N) | Flash combustion (Carlo Erba Instruments, Milan, Italy) |
| | (AOAC, 1990) |
| Neutral detergent fiber | ANKOM^{200/220} Fiber Analyzer (ANKOM Technology, Fairport, |
| (NDF) | NY) according to the methodology supplied by the company (based |
| | Van Soest et al., 1991), |
| | Sodium sulphite and heat-stable amylase used |
| Acid detergent fiber (ADF) | ANKOM^{200/220} Fiber Analyzer (ANKOM Technology, Fairport, |
| | NY) according to the methodology supplied by the company (based |
| | on Van Soest et al., 1991) |
| Starch | Enzymatic hydrolysis of α-linked glucose polymers |
| | (Rode et al., 1999) |
| Yb | Atomic absorption (AOAC, 1990) |
| Enzyme Activities | |
| Xylanase activity | Substrate was birchwood xylan in 0.1 M citrate phosphate buffer |
| | (pH 6.0; 10 mg/ml). 40 μL of strained ruminal fluid was incubated |
| | with 1 ml of substrate. Incubations were performed in triplicate for |
| | 60 min. The enzymatic reaction was terminated by adding |
| | dinitrosalicylic acid reagent. The reaction contents were boiled for |
| | 15 min. and cooled in cold water. Absorbance was read at 530 nm |
| | using MRX-HD plate reader. These values were converted to |
| | reducing sugars using xylose standard. Blanks, substrate alone and |
| | enzyme alone were used to correct for substrate autolysis and |
| | sugars in the enzyme sample, respectively. One unit of activity was |
| | defined as the amount of enzyme required to release 1 nmol of |
| | xylose/min. |
| Carboxymethylcellulase | Substrate was medium-viscosity carboxymethylcellulose (Sigma |
| activity (CMC) | Chemicals, St. Louis, MO). Analysis was the same as for xylanase |
| | except for incubations for 120 min at 39° C. Absorbance values |
| | were converted to reducing sugars using standard glucose curves. |
| | One unit of activity was defined as the amount of enzyme required |
| | to release 1 nmol of glucose/min. |
| Exoglucanase, | Performed using stock solutions (1 mM) of derivatives: p-NP-β-D- |
| β-D-glucosidase, | cellobioside, p-NP-β-D-glucopyranoside, p-NP-β-D-xylopyranoside, |
| β-D-xylosidase, | and p-NP-α-L-arabinofuranoside, respectively. Samples of strained |
| arabinofuranosidase | ruminal fluid (20 μl) were incubated with 80 μl of substrate |
| | (prepared in 0.1 M citrate phosphate buffer, pH 6.0) at 39° C. for 60 min. |
| | The reaction was terminated by the addition of 100 μl of 1 M |
| | glycine-NaOH buffer (pH 10.8). The release of p-nitrophenol was |
| | determined colorimetrically at 420 nm. One unit of each enzyme |
| | activity was defined as the amount of enzyme required to release 1 nmol |
| | of p-nitrophenol/min. |
| Protease activity | Assayed using azocasein (lot 25H7125, Sigma Chemical, St. Louis, |
| | MO) as a substrate in a similar manner by Brock et al. (1982). |
| | Strained ruminal fluid (0.4 ml) was added to 0.5 ml of azocasein |
| | (2% wt/vol) in 0.1 M citrate phosphate buffer (pH 6.8). Triplicate |
| | tubes were mixed and incubated for 1 h at 39° C. Reactions were |
| | stopped by the addition of 0.5 ml of 15% (wt/vol) trichloroacetatic |
| | acid (TCA). Background controls, in which azocasein was added |
| | after reactions were terminated with TCA, were also included. |
| | After addition of TCA, tubes were mixed, placed on ice for 30 min. |
| | and then centrifuged at 15,600 × g for 5 min at room temperature. |
| | Supernatant (0.75 ml) was mixed with 0.75 ml of 0.5 M NaOH and |
| | absorbance was measured spectrophotometrically at 420 nm using |
| | MRX-HD |
| | plate reader. |

h. Statistical Analyses

All data were statistically analyzed using the mixed model procedure in SAS™ (SAS Institute, 1999, Cary, N.C.). Data digestibility were analyzed using a model that accounted for the fixed effect of square (i.e., non-cannulated vs. cannulated cows), fixed effect of level of forage in the diet (i.e., high vs. low forage), fixed effect of enzyme (i.e., non-protease vs. protease), fixed effect of the interaction between the forage and enzyme, the random effect of cow within square, the random effect of period within square, and the residual error. Data for ruminal enzyme activities were analyzed with the same model but by also accounting for the repeated measures. Differences were considered significant at P<0.05.

Table 18 shows that adding the protease enzyme to the diet increased the digestibility of the diet. Digestibility of DM, OM, N, ADF, and NDF were consistently increased due to protease enzyme. The magnitude of improvement in digestibility was generally greater for the low forage diet than for the higher forage diet, but for both diets the improvements in digestibility were substantial.

Table 19 shows that by adding protease enzyme to the diet, the enzyme activities in ruminal fluid were increased. In particular, activities of xylanase, endoglucanase, and protease were increased. Because the enzyme product contained no measurable xylanase or endoglucanase activity, the higher activities in ruminal fluid had to be the result of increased micobial activity. These data clearly show that adding a protease enzyme to the diet of do cows increased the overall fibrolytic activity within the rumen. Thus, adding protease caused a synergy with the microbial population. An increase in the fiber-digesting capacity of the rumen would account for the increase in feed digestion presented in Table 18.

### EXAMPLE 6

### Effects of Protease Enzyme on In Vitro Digestibility of Forage

This study was conducted using the forages from the in vivo study in Example 5. The study was conducted to determine the effects of adding a protease enzyme product on forage digestibility measured in vitro.

In vitro ruminal gas production of forages was measured using a system similar to that described by Mauricio et al. (1999). Fresh samples of the alfalfa hay and barley silage that were used in the in vivo experiment described in Example 5 were milled for 10 seconds using a Knifetec™ 1095 sample mill (Foss Tecator, Höganäs, Sweden). Samples of the milled forages approximately equal to 1 g of DM were then weighed into gas-tight serum culture vials (125 ml capacity) with eight replications. The same commercially available protease product used in Example 5 (Protex 6L® Genencor International, Rochester, N.Y.) was used. The enzyme was applied at a rate of 1.25 μl/g DM forage 20 hours prior to inoculation with ruminal fluid which is the same application rate that was used in Example 5. Three hours after the enzyme was added to the tubes, 40 ml of anaerobic buffer medium, prepared as outlined by Goering and Van Soest (1970) and adjusted to pH 6.0 using 1 M trans-aconitic acid (Sigma Chemicals) was added, and the vials were stored at 20° C. overnight.

Ruminal fluid was obtained 4 hours post feeding (1100 h) from a lactating dairy cow fed a diet composed of barley silage, chopped alfalfa hay, rolled corn grain, and concentrate. Strained ruminal fluid collected as described for Example 5 was transported to the laboratory in sealed, preheated containers and was kept at 39° C. in a water bath. The inoculum was dispensed (10 ml per vial) into culture vials which had been warmed to 39° C. in an incubator and flushed with oxygen-free CO₂. The vials were then sealed with a 14 mm butyl rubber stopper plus aluminium crimp cap immediately after loading and were incubated for 48 h. Negative controls (ruminal fluid plus buffer alone and ruminal fluid plus buffer and enzyme product) were also incubated in eight replications. These controls were used to correct for gas release and fermentation residues resulting directly from the inoculum. Headspace gas produced by substrate fermentation was measured at 2, 4, 6, 8, 10, 12, 18, 24, 30, 36, 42, and 48 hours post inoculation by inserting a 23 gauge (0.6 mm) needle attached to a pressure transducer (type T443A, Bailey and Mackey, Birmingham, UK) connected to a visual display (Data Track, Christchurch, UK). The transducer was then removed leaving the needle in place to permit venting. Pressure values, corrected by the amount of substrate organic matter incubated and for gas release from negative controls, were used to generate volume estimates using the equation (gas volume=0.18+3697×gas pressure+0.0924×gas pressure²) reported by Mauricio et al. (1999). On removal, the vials were placed in the refrigerator at 4° C. for 2 hours to stop fermentation, and filtered.

Table 20 shows that adding protease to alfalfa hay increased the gas production starting at 2 hours of incubation, and the increase was maintained throughout the incubation. Increased gas production indicates an improvement in microbial digestion. In contrast, adding protease had no effect on the gas production of barley silage.

### REFERENCES

Beauchemin, K. A., Morgavi, D. P., McAllister, T. A., Yang, W. Z. and Rode, L. M. (2001) The use of feed enzymes in ruminant diets. In Recent Advances in Animal Nutrition. P. C. Garnsworthy and P. J. Wiseman, eds. Nottingham University Press, Nottingham, UK

Brown, R. L., Chen, Z. Y., Cleveland, T. E., Cotty, P. J. and Cary, J. W. (2001) Variation in in vitro α-amylase and protease activity is related to the virulence of Aspergillus flavus isolates. J. Food Prod. 64;401-404.

Bryant, M. P. and Burkey, L. A. (1953) Cultural methods and some characteristics of some of the more numerous groups of bacteria in the bovine rumen. J. Dairy Sci. 36:205-207.

Colombatto, D. (2000) Use of enzymes to improve fibre utilization in ruminants: a biochemical and in vitro rumen degradation assessment. PhD. Thesis. The University of Reading. Reading, UK.

Colombatto, D., Morgavi, D. P., Furtado, A. F. and Beauchemin, K. A. (2003) Screening of fibrolytic enzymes as additives for ruminant diets: relationship between enzyme activities and the in vitro degradation of enzyme-treated forages. Proc. Brit. Soc. Anim. Sci. BSAS, York, UK, p. 210.

Colombatto, D. Mould. F. L., Bhat. M. K., Morgavi, D. P., Beauchemin, K. A. and Owen, E. (2003) Influence of fibrolytic enzymes on the hydrolysis and fermentation of pure cellulose and xylan by mixed ruminal microorganisms in vitro. Proc. Brit. Soc. Anim. Sci. BSAS, York, UK, p. 208.

Dehority, B. A., Tirabasso, P. A. and Grifo Jr., A. P. (1989) Most probable-number procedures for enumerating ruminal bacteria, including the simultaneous estimation of total and cellulolytic numbers in one medium. Appl. Environ. Microbiol. 55:2789-2792.

Goering, H. K. and Van Soest, P. J. (1970) Forage Fiber Analyses: Apparatus, Reagents, Procedures and Some Applications. Agri. Handbook No. 379, ARS-USDA, Washington. D.C.

Hoover, W. H., Miller, T. K., Stokes, S. R. and Thayne, W. V. (1989) Effects of fish meals on ruminal bacterial fermentation in continuous culture. J. Dairy Sci. 72:2991-2998.

Mauricio, R. M., Mould, F. L., Dhanoa, M. S., Owen, E., Channa, K. S. and Theodorou, M. K. (1999) A semi-automated in vitro gas production technique for ruminant feedstuff evaluation. Anim. Feed Sci. Technol. 79:321-330.

McDougall, E. I. (1948) Studies on ruminant saliva 1. The composition and output of sheep's saliva Biochem. J. 43:99-109.

Nsercko, V. L., Morgavi, D. P., Rode, L. M., Beauchemin, K. A. and McAllister, T. A. (2000) Effects of fungal enzyme preparations on hydrolysis and subsequent degradation of alfalfa hay fiber by mixed rumen microorganisms in vitro. Anim. Feed Sci. Technol 88:153-170.

Rode, L. M., Yang, W. Z. and Beauchemin, K. A. (1999) Fibrolytic enzyme supplements for dairy cows in early lactation. J. Dairy Sci. 82:2121-2126.

Van Soest, P. J., Robertson, J. B. and Lewis, B. A. (1991) Methods for dietary fiber, neutral detergent fiber, and nonstarch polysaccharides in relation to animal nutrition. J. Dairy Sci. 74:3583-3597.

Van Soest, P. J. (1994) Nutritional Ecology of the Ruminant. Cornell University Press, Ithaca, N.Y.

Verdouw, H. (1978) Ammonia determination based on indophenol formation with sodium salicylate. Water Res. 12:399-402.

Wood. T. M. and Bhat. M. K. (1988) Methods for measuring cellulase activities. In Methods of Enzymology. W. A. Wood and S. T. Kellogg, eds. Academic Press, Inc., London, UK, pp. 87-112.

Yang, W. Z., Beauchemin, K. A. and Rode, L. M. (1999) Effects of an enzyme feed additive on extent of digestion and milk production of lactating dairy cows. J. Dairy Sci. 82:391-403.

### PATENT DOCUMENTS

Beauchemin, K. A., Rode, L. and Sewalt, V. J. Enzyme additives for ruminant feeds. U.S. Pat. No. 5,720,971, issued Feb. 24, 1998.

All publications mentioned in this specification are indicative of the level of skill in the art to which this invention pertains. All publications are herein incorporated by reference to the same extent as if each individual publication was specifically and individually indicated to be incorporated by reference.

Although the foregoing invention has been described in some detail by way of illustration and example, for purposes of clarity and understanding it will be understood that certain changes and modifications may be made without departing from the scope or spirit of the invention as defined by the following claims.

TABLE 1
Protein concentrations (mg/mL), enzymic activities (μmol sugar or
p-nitrophenol min⁻¹ g⁻¹ or mm) and reducing sugars
released (mg) from the incubation of alfalfa hay (AH) and
corn silage (CS) with the enzyme products

| | | Enzymic activity^{x} | | | | | | | | |
|---|---|---|---|---|---|---|---|---|---|---|
| Product | Protein | OSX | BX | CMC | SCELL | BG | XG | AGAL | LICH | AX |
| RT1180 | 114 | 222 | 143 | 591 | 32 | 535 | 317 | 0.0 | 832 | 108 |
| RT1181 | 116 | 81 | 0.8 | 332 | 74 | 351 | 199 | 0.0 | 560 | 22 |
| RT1182 | 37 | 3228 | 3160 | 72 | 0.0 | 6.4 | 34 | 0.0 | 57 | 915 |
| RT1183 | 109 | 487 | 517 | 421 | 29 | 279 | 178 | 0.0 | 419 | 364 |
| RT1184 | 128 | 28 | 6.7 | 0.0 | 0.5 | 53 | 2.1 | 0.0 | 71 | 2.6 |
| RT1185 | 90 | 84 | 58 | 324 | 38 | 264 | 329 | 0.0 | 639 | 79 |
| RT1186 | 120 | 429 | 336 | 663 | 15 | 526 | 242 | 0.0 | 647 | 177 |
| RT1187 | 119 | 138 | 49 | 419 | 60 | 351 | 193 | 0.0 | 431 | 0.0 |
| RT1188 | 121 | 145 | 47 | 356 | 26 | 310 | 132 | 0.0 | 345 | 0.0 |
| RT1189 | 101 | 323 | 228 | 493 | 32 | 376 | 249 | 0.0 | 560 | 33 |
| RT1190 | 467 | 1116 | 517 | 71 | 62 | 101 | 19 | 5.6 | 55 | 461 |
| RT1191 | 795 | 340 | 119 | 897 | 181 | 829 | 479 | 0.0 | 1464 | 87 |
| RT1192 | 754 | 524 | 347 | 1047 | 123 | 875 | 774 | 0.0 | 1618 | 398 |
| RT1193 | 259 | 74 | 17 | 88 | 0.0 | 16 | 38 | 0.0 | 32 | 132 |
| RT1194 | 371 | 566 | 91 | 114 | 8.4 | 40 | 77 | 47 | 197 | 148 |
| RT1195 | 80 | 51 | 68 | 25 | 0.0 | 57 | 6.4 | 0.0 | 40 | 2.7 |
| RT1196 | 315 | 2928 | 2063 | 178 | 0.0 | 710 | 42 | 0.0 | 790 | 857 |
| RT1197 | 546 | 2000 | 1466 | 713 | 164 | 1591 | 166 | 121 | 2076 | 871 |
| RT1198 | 72 | 296 | 47 | 0.2 | 0.0 | 115 | 209 | 0.0 | 442 | 43 |
| RT1199 | 65 | 95 | 85 | 75 | 66 | 5.6 | 23 | 190 | 46 | 66 |
| RT1200 | 233 | 536 | 542 | 351 | 81 | 315 | 390 | 0.0 | 807 | 359 |
| RT1201 | 179 | 2686 | 1945 | 21 | 0.2 | 4.8 | 0.0 | 0.0 | 16 | 518 |
| Promote^{z} | 85 | 2720 | 2252 | 225 | 24 | 120 | 64 | 0.0 | 212 | 1236 |

| | |
|---|---|
| Enzymic activity^{x} | RS^{y} |

| Product | AMYL | LAM | ES | AF | GPY | XPY | PRT | GAPY | AH | CS |
|---|---|---|---|---|---|---|---|---|---|---|
| RT1180 | 14 | 0.0 | 8.6 | 0.7 | 11 | 3.0 | 7.3 | 2.8 | 1.2 | 0.7 |
| RT1181 | 5.6 | 0.0 | 3.4 | 0.3 | 6.0 | 0.3 | 3.6 | 0.0 | 1.0 | 0.8 |
| RT1182 | 7.6 | 0.0 | 34 | 0.4 | 4.5 | 5.0 | 7.2 | 1.0 | 0.7 | 0.2 |
| RT1183 | 12 | 0.4 | 13 | 0.5 | 3.5 | 1.3 | 6.3 | 0.1 | 0.9 | 0.8 |
| RT1184 | 7.9 | 0.0 | 12 | 0.0 | 0.0 | 0.0 | 29.6 | 0.0 | 0.5 | 0.0 |
| RT1185 | 10 | 2.3 | 4.0 | 0.3 | 3.1 | 0.3 | 7.8 | 0.0 | 0.5 | 0.7 |
| RT1186 | 10 | 31 | 3.6 | 0.6 | 15 | 3.2 | 0.0 | 1.8 | 0.7 | 1.2 |
| RT1187 | 3.3 | 23 | 3.6 | 0.1 | 5.0 | 0.4 | 0.0 | 0.0 | 0.6 | 1.1 |
| RT1188 | 13 | 10 | 8.0 | 0.4 | 4.3 | 0.9 | 8.4 | 0.0 | 0.4 | 0.6 |
| RT1189 | 2.1 | 22 | 8.8 | 0.3 | 10 | 2.3 | 5.6 | 2.0 | 0.6 | 0.8 |
| RT1190 | 1586 | 199 | 25 | 0.1 | 10 | 0.3 | 30.1 | 6.3 | 11 | 1.5 |
| RT1191 | 28 | 69 | 11 | 1.0 | 12 | 0.4 | 0.0 | 0.0 | 10 | 3.9 |
| RT1192 | 32 | 23 | 16 | 0.6 | 18 | 1.9 | 0.0 | 0.0 | 8.4 | 2.9 |
| RT1193 | 283 | 129 | 6.0 | 1.2 | 4.3 | 3.3 | 21.0 | 2.2 | 5.6 | 0.4 |
| RT1194 | 1892 | 138 | 3.1 | 0.1 | 2.0 | 0.1 | 24.5 | 5.1 | 3.6 | 0.7 |
| RT1195 | 31 | 35 | 0.5 | 0.0 | 0.0 | 0.0 | 0.0 | 0.0 | 2.0 | 0.0 |
| RT1196 | 1068 | 23 | 58 | 11 | 8.6 | 18 | 13.4 | 8.0 | 0.9 | 2.9 |
| RT1197 | 694 | 174 | 103 | 3.7 | 58 | 13 | 16.7 | 1.9 | 0.6 | 2.2 |
| RT1198 | 293 | 14 | 20 | 16 | 2.2 | 1.4 | 12.1 | 4.7 | 0.5 | 0.7 |
| RT1199 | 9.3 | 119 | 26 | 52 | 1.4 | 0.0 | 8.6 | 13 | 2.1 | 0.4 |
| RT1200 | 33 | 40 | 71 | 17 | 3.8 | 29 | 7.8 | 0.0 | 1.3 | 0.9 |
| RT1201 | 465 | 82 | 0.0 | 0.0 | 0.0 | 0.0 | 7.4 | 0.0 | 1.7 | 2.7 |
| Promote^{z} | 9.1 | 43 | 7.5 | 0.5 | 15 | 0.6 | 20.0 | 0.0 | ND | ND |

^{x}OSX = xylanase (oat spelt); BX = xylanase (birchwood); CMC = endoglucanase; SCELL = exoglucanase; BG = β-glucanase; XG = xyloglucan; AGAL = arabinogalactan; LICH = lichenase; AX = arabinoxylan; AMYL = α-amylase; LAM = laminarinase; ES = esterase; AF = arabinofuranosidase; GPY = β-glucosidase; XPY = β-xylosidase; PRT = protease; GAPY = galactosidase.
^{y}RS = reducing sugars, determined after incubation of 25 mg freeze-dried substrate at 39° C. for 15 min with each enzyme product in triplicate.
^{z}Promote N.E.T. (Lot MO-E100) was used.

TABLE 2
Relationship between enzymic activities and the release of
reducing sugars from alfalfa hay and corn silage

| | | Partial | Model | | |
|---|---|---|---|---|---|
| Forage | Variable | R² | R² | F value | P > F |
| Alfalfa | Protein content (x) | 0.60 | | 30.6 | <0.001 |
| hay | β-Glucanase (y) | 0.24 | 0.84 | 28.3 | <0.001 |
| Equa- | Sugar = 0.017 x − 0.029 y + 0.2897 | | | | |
| tion | | | | | |
| Corn | Protein content (a) | 0.59 | | 29.4 | <0.001 |
| silage | Xylanase (oat spells) (b) | 0.09 | 0.68 | 5.4 | 0.031 |
| | Endoglucanase (c) | 0.05 | 0.74 | 3.8 | 0.067 |
| | Xylanase (birchwood) (d) | 0.09 | 0.83 | 8.6 | 0.009 |
| | α-Amylase (e) | 0.09 | 0.91 | 16.3 | 0.001 |
| | β-Glucosidase (f) | 0.04 | 0.95 | 12.3 | 0.003 |
| | Exoglucanase (g) | 0.01 | 0.96 | 4.8 | 0.047 |

| | |
|---|---|
| Equa- | Sugar = 0.001 a + 0.027 b + 0.009 c − 0.026 d − |
| tion | 0.005 e − 0.032 f + 0.025 g − 0.2251 |

TABLE 3
Effects of enzyme addition (1.5 μL/g DM)
on the apparent DMD (g/kg) of alfalfa hay
or corn silage after 18 h of incubation with ruminal fluid

| Treatment | Alfalfa hay | Ranking^{w} | Corn silage | Ranking^{w} |
|---|---|---|---|---|
| Control | 434.9 | 23 | 424.0 | 24 |
| RT1180 | 450.4 | 18 | 438.0 | 19 |
| RT1181 | 431.8 | 24 | 452.4^{y} | 7 |
| RT1182 | 462.2 | 8 | 439.1 | 18 |
| RT1183 | 459.4 | 10 | 462.7^{z} | 2 |
| RT1184 | 477.4^{y} | 2 | 443.7 | 12 |
| RT1185 | 454.3 | 15 | 441.6 | 16 |
| RT1186 | 449.2 | 19 | 455.8^{z} | 5 |
| RT1187 | 457.0 | 14 | 461.0^{z} | 3 |
| RT1188 | 459.0 | 11 | 443.0 | 15 |
| RT1189 | 454.3 | 16 | 454.8^{z} | 6 |
| RT1190 | 472.0^{y} | 5 | 448.8^{y} | 10 |
| RT1191 | 467.5 | 7 | 447.9^{y} | 11 |
| RT1192 | 462.2 | 9 | 448.9^{y} | 9 |
| RT1193 | 458.9 | 12 | 437.7 | 20 |
| RT1194 | 443.5 | 22 | 432.9 | 21 |
| RT1195 | 444.9 | 21 | 419.2 | 26 |
| RT1196 | 475.4^{y} | 4 | 432.4 | 22 |
| RT1197 | 468.8 | 6 | 423.8 | 25 |
| RT1198 | 458.8 | 13 | 443.4 | 13 |
| RT1199 | 452.9 | 17 | 449.0 | 8 |
| RT1200 | 445.2 | 20 | 443.1 | 14 |
| RT1201 | 479.7^{y} | 1 | 424.2 | 23 |
| Promote N.E.T. | 476.1^{y} | 3 | 439.6 | 17 |
| Promote Dairy | ND^{x} | ND | 470.2^{z} | 1 |
| Promote Beef | ND | ND | 459.0^{z} | 4 |
| SEM | 24.56 | | 7.96 | |

^{w}Relative ranking according to DMD.
^{x}ND = not determined.
^{y,z}Different from the control at P < 0.05 and P < 0.01, respectively.

TABLE 4
Relationship between enzymic activities (μmol xylose min⁻¹ g⁻¹)
and the apparent DMD (g/kg) of alfalfa hay and corn silage,
after 18 h of incubation with ruminal fluid

| Forage | Enzymic activity | Regression Equation | Partial R² | Model R² | P > F |
|---|---|---|---|---|---|
| Alfalfa hay | Xylanase (oat spelts) | DMD = 0.04 x + 0.41 y + 449.9 | 0.29 | | 0.010 |
| | Protease | | 0.10 | 0.39 | 0.096 |
| Corn silage | Xylanase (oat spelts) | DMD = −0.033 x + 446.6 | | 0.19 | 0.044 |

TABLE 5
Dry matter degradation (g/kg) kinetics of alfalfa hay
or corn silage, untreated or treated with enzyme
products at 1.5 μL/g DM

| | Incubation time, h | | | | | |
|---|---|---|---|---|---|---|
| Treatment | 0 | 6 | 18 | 30 | 48 | 96 |
| Alfalfa hay | | | | | | |
| Control | 307.0 | 409.6^{a} | 575.9 | 690.6 | 745.2 | 765.9 |
| Promote Dairy | 313.3 | 386.8^{a} | 573.0 | 680.2 | 741.9 | 762.6 |
| RT1184 | 334.0 | 446.3^{b} | 609.0 | 689.4 | 744.8 | 769.7 |
| RT1197 | 319.2 | 410.6^{a} | 568.8 | 680.2 | 739.2 | 769.9 |
| SEM | 14.14 | 23.13 | 25.08 | 24.19 | 11.06 | 7.53 |
| Corn silage | | | | | | |
| Control | 294.1 | 318.2^{a} | 455.3^{ab} | 521.6 | 619.2^{a} | 764.3^{ab} |
| Promote Dairy | 288.1 | 322.7^{ab} | 435.9^{a} | 523.4 | 648.7^{b} | 754.8^{ab} |
| RT1181 | 307.9 | 344.5^{b} | 476.4^{b} | 549.3 | 641.6^{b} | 767.1^{b} |
| RT1183 | 279.7 | 318.7^{a} | 451.6^{ab} | 527.5 | 634.7^{b} | 752.8^{a} |
| SEM | 29.79 | 38.61 | 29.09 | 24.39 | 10.14 | 13.05 |

^{a,b}Within substrates and columns, means without common superscripts differ (P < 0.05).

TABLE 6
Fiber degradation kinetics of alfalfa hay, untreated or
treated with enzyme products at 1.5 μL/g DM

| | Incubation time, h | | | | | |
|---|---|---|---|---|---|---|
| Treatment | 0 | 6 | 18 | 30 | 48 | 96 |
| NDF, g/kg | | | | | | |
| Control | −12.3 | 34.0^{ab} | 317.6 | 365.8 | 454.6 | 493.0 |
| Promote Dairy | 5.8 | 19.6^{a} | 188.2 | 343.6 | 452.0 | 490.7 |
| RT1184 | 12.3 | 51.3^{b} | 240.1 | 354.9 | 449.8 | 509.0 |
| RT1197 | −4.7 | 53.7^{b} | 198.0 | 343.3 | 454.0 | 500.8 |
| SEM | 27.25 | 22.53 | 38.01 | 33.27 | 22.68 | 16.37 |
| ADF, g/kg | | | | | | |
| Control | −8.8 | −3.0 | 153.5 | 322.4 | 408.0 | 440.5 |
| Promote Dairy | 0.1 | −25.9 | 116.3 | 289.9 | 407.3 | 436.3 |
| RT1184 | 12.2 | −20.2 | 167.4 | 303.4 | 407.4 | 452.9 |
| RT1197 | −7.5 | 16.6 | 130.2 | 300.3 | 417.0 | 461.0 |
| SEM | 34.36 | 27.12 | 35.35 | 36.63 | 23.04 | 19.30 |
| Hemicellulose, | | | | | | |
| g/kg | | | | | | |
| Control | −19.2 | 106.1^{a} | 342.6 | 450.4 | 545.5 | 595.1 |
| Promote Dairy | 17.1 | 108.4^{a} | 328.4 | 448.2 | 539.0 | 596.4 |
| RT1184 | 12.5 | 190.8^{b} | 381.9 | 455.2 | 532.5 | 606.1 |
| RT1197 | 0.6 | 125.8^{ab} | 329.9 | 426.9 | 526.1 | 578.3 |
| SEM | 20.41 | 23.55 | 45.47 | 29.59 | 24.79 | 16.62 |

^{a,b}Within fractions and columns, means without common superscripts differ (P < 0.05).

TABLE 7
Fiber degradation kinetics of corn silage, untreated or
treated with enzyme products at 1.5 μL/g DM

| | Incubation time, h | | | | | |
|---|---|---|---|---|---|---|
| Treatment | 0 | 6 | 18 | 30 | 48 | 96 |
| NDF, g/kg | | | | | | |
| Control | 5.7 | 17.5 | 116.1^{a} | 193.5 | 327.3^{a} | 581.0^{ab} |
| Promote Dairy | 29.2 | 39.4 | 119.3^{a} | 196.6 | 380.0^{b} | 560.3^{a} |
| RT1181 | 44.3 | 45.3 | 147.0^{b} | 225.7 | 368.7^{b} | 587.5^{b} |
| RT1183 | 18.2 | 17.7 | 104.2^{a} | 184.2 | 354.6^{ab} | 565.1^{a} |
| SEM | 14.88 | 12.53 | 19.26 | 15.74 | 13.75 | 19.85 |
| ADF, g/kg | | | | | | |
| Control | −4.2 | 14.9 | 78.0^{a} | 153.6 | 292.1^{a} | 553.7 |
| Promote Dairy | 13.3 | 21.6 | 79.7^{a} | 159.1 | 345.6^{b} | 527.6 |
| RT1181 | 42.0 | 48.2 | 111.7^{b} | 204.6 | 333.5^{b} | 554.8 |
| RT1183 | 4.6 | 17.1 | 72.5^{a} | 154.8 | 321.5^{ab} | 541.8 |
| SEM | 19.85 | 23.05 | 12.99 | 17.56 | 12.97 | 27.19 |
| Hemicellulose, g/kg | | | | | | |
| Control | 17.6 | 20.5^{a} | 161.5^{b} | 241.1 | 369.1^{a} | 613.7^{bc} |
| Promote Dairy | 48.1 | 60.6^{c} | 166.4^{b} | 241.4 | 421.0^{b} | 599.3^{ab} |
| RT1181 | 47.1 | 41.8^{b} | 189.0^{b} | 250.8 | 410.6^{ab} | 626.4^{c} |
| RT1183 | 34.4 | 18.4^{a} | 142.0^{a} | 219.2 | 393.8^{ab} | 592.8^{a} |
| SEM | 10.27 | 2.39 | 27.12 | 14.44 | 15.63 | 12.15 |

^{a,b,c}Within fractions and columns, means without common superscripts differ (P < 0.05).

TABLE 8
Degradation profiles (g/kg DM) of the non-fiber fractions (100-NDF),
and percentage of increase in DMD for treatments RT1184
and RT1181 attributable to NDF degradation

| Sub- | | | | | | | |
|---|---|---|---|---|---|---|---|
| strate | Treatment | Incubation time, h | | | | | |
| | | 0 | 6 | 18 | 30 | 48 | 96 |
| Alfalfa | Control | 311.7 | 396.6 | 492.8 | 550.9 | 571.5 | 577.6 |
| hay | Promote Dairy | 311.1 | 379.3 | 501.1 | 548.9 | 569.2 | 575.2 |
| | RT1184 | 329.3 | 426.7 | 517.3 | 553.8 | 573.0 | 575.3 |
| | RT1197 | 321.0 | 390.1 | 493.2 | 549.1 | 565.8 | 578.6 |
| Increase in DMD, % | | 8.8 | 9.0 | 5.7 | −0.2 | −0.05 | 0.5 |
| Increase in DMD due | | 34.8 | 18.0 | 25.9 | 0 | 0 | 100 |
| to NDF, % | | | | | | | |
| Corn | Control | 291.4 | 310.0 | 401.0 | 431.2 | 466.2 | 492.7 |
| silage | Promote Dairy | 274.5 | 304.3 | 380.1 | 431.5 | 471.1 | 492.9 |
| | RT1181 | 287.2 | 323.3 | 407.7 | 443.8 | 469.3 | 492.5 |
| | RT1183 | 271.2 | 310.4 | 402.9 | 441.4 | 469.0 | 488.7 |

| | | | | | | |
|---|---|---|---|---|---|---|
| Increase in DMD, % | 4.7 | 8.3 | 4.6 | 5.3 | 3.6 | 0.4 |
| Increase in DMD due to | 100 | 49.4 | 68.4 | 54.3 | 86.4 | 100 |
| NDF, % | | | | | | |

TABLE 9
Dry matter degradation (g/kg) kinetics of a mixture
of alfalfa hay and corn silage,
untreated or treated with enzyme products

| | Incubation time, h | | | | | |
|---|---|---|---|---|---|---|
| Treatment^{y} | 0 | 6 | 18 | 30 | 48 | 96 |
| Control | 319.1^{a} | 411.1^{a} | 563.4^{a} | 632.8 | 709.4 | 774.5^{a} |
| RT1181 | 322.0^{a} | 424.3^{ab} | 564.3^{a} | 649.5 | 724.6 | 774.9^{a} |
| RT1184 | 354.4^{b} | 445.2^{b} | 592.4^{b} | 657.2 | 739.8 | 782.9^{b} |
| 8184Low | 336.2^{ab} | 410.8^{a} | 558.9^{a} | 637.1 | 720.1 | 781.4^{b} |
| 8184High | 336.8^{ab} | 442.0^{ab} | 579.4^{ab} | 655.5 | 733.9 | 783.4^{b} |
| SEM | 12.91 | 11.67 | 7.83 | 10.55 | 11.33 | 1.45 |

^{a,b}Within columns, means without common superscripts differ (P < 0.05).
^{y}Control = no enzyme added; RT1181 and RT1184 = enzymes added at 1.5 μL/g DM; 8184Low = a mixture (1:1) of RT1181 and RT1184 added at 0.5 μL/g DM; 8184High = a mixture (1:1) of RT1181 and RT1184 added at 1.5 μL/g DM.

TABLE 10
Fiber degradation (g/kg) kinetics of a mixture of alfalfa
hay and corn silage, untreated or treated with enzyme products

Incubation time, h

| Treatment^{y} | 0 | 6 | 18 | 30 | 48 | 96 |
|---|---|---|---|---|---|---|
| NDF, g/kg | | | | | | |
| Control | −36.8 | 21.0^{a} | 179.0^{ab} | 277.5 | 399.1 | 526.0^{a} |
| RT1181 | −27.6 | 32.8^{ab} | 164.6^{a} | 303.0 | 427.3 | 525.4^{a} |
| RT1184 | −13.5 | 50.9^{bc} | 215.5^{b} | 317.9 | 444.6 | 536.6^{b} |
| 8184Low | −7.1 | 26.4^{a} | 180.5^{ab} | 287.1 | 418.2 | 544.8^{c} |
| 8184High | −14.0 | 59.6^{c} | 207.9^{b} | 318.7 | 440.1 | 544.7^{c} |
| SEM | 5.45 | 7.03 | 11.24 | 19.35 | 24.68 | 3.39 |
| ADF, g/kg | | | | | | |
| Control | −20.2 | −10.2 | 119.2 | 225.1 | 354.7 | 487.9^{ab} |
| RT1181 | −21.0 | 10.2 | 111.4 | 258.1 | 380.3 | 480.5^{a} |
| RT1184 | −10.0 | 16.4 | 155.1 | 263.9 | 409.3 | 499.4^{ab} |
| 8184Low | 6.2 | −19.6 | 118.4 | 234.5 | 370.5 | 504.1^{bc} |
| 8184High | −12.8 | 18.4 | 150.8 | 265.7 | 398.7 | 508.3^{c} |
| SEM | 7.49 | 15.14 | 15.45 | 16.22 | 24.81 | 5.56 |
| Hemicellulose, | | | | | | |
| g/kg | | | | | | |
| Control | −61.8 | 67.7^{a} | 268.5^{ab} | 355.9 | 465.5 | 583.0^{a} |
| RT1181 | −37.4 | 66.5^{a} | 244.4^{a} | 370.2 | 497.6 | 592.6^{ab} |
| RT1184 | −18.9 | 102.6^{ab} | 305.1^{b} | 398.5 | 497.3 | 592.4^{ab} |
| 8184Low | −27.2 | 95.4^{ab} | 273.4^{ab} | 365.7 | 489.6 | 605.7^{b} |
| 8184High | −15.9 | 121.2^{b} | 293.3^{b} | 398.1 | 501.9 | 599.2^{ab} |
| SEM | 10.95 | 15.93 | 20.47 | 28.22 | 30.73 | 12.29 |

^{a,b,c}Within fractions and columns, means without common superscripts differ (P < 0.05).
^{y}Control = no enzyme added; RT1181 and RT1184 = enzymes added at 1.5 μL/g DM; 8184Low = a mixture (1:1) of RT1181 and RT1184 added at 0.5 μL/g DM; 8184High = a mixture (1:1) of RT1181 and RT1184 added at 1.5 μL/g DM.

TABLE 11
Chemical composition (g/kg DM) of the feeds
and of the total mixed ration (TMR)

| | Feed | | | |
|---|---|---|---|---|
| | Alfalfa hay | Corn silage | Rolled corn | TMR^{a} |
| DM | 904.2 | 416.9 | 883.3 | 643.6 |
| OM | 885.1 | 946.1 | 985.7 | 943.6 |
| CP | 232.6 | 113.3 | 100.8 | 142.9 |
| NDF | 433.3 | 369.1 | 131.5 | 321.7 |
| ADF | 284.1 | 177.8 | 23.9 | 166.8 |
| ADL | 58.6 | 8.5 | 0.0 | 26.3 |
| Starch | 14.3 | 277.2 | 575.2 | 286.3 |

^{a}The total mixed ration was composed (DM basis) of 30% alfalfa hay, 30% corn silage, and 40% rolled corn.

TABLE 12
Effects of pH and enzyme addition on total microbial
(TMC) and cellulolytic bacteria counts (CBP) in continuous
culture at 6 h post feed provision to the fermenters

| | | | | | | Effects, P < | | |
|---|---|---|---|---|---|---|---|---|
| | Treatment^{a} | | | | | | | pH × |
| | HC | HT | LC | LT | SEM | pH | Enzyme | Enzyme |
| TMC, | 9.04 | 9.21 | 9.30 | 9.41 | 0.092 | 0.03 | 0.13 | 0.70 |
| Log₁₀ | | | | | | | | |
| CBP, | 3.65 | 4.16 | 3.00 | 2.59 | 0.389 | 0.01 | 0.88 | 0.16 |
| Log₁₀ | | | | | | | | |

^{a}HC = high pH with control TMR; HT = high pH with TMR treated with enzymes; LC = low pH with control TMR; LT = low pH with TMR treated with enzymes.

TABLE 13
Effects of pH and enzyme addition on enzymic activities at 6 h post-feeding

| | Treatment^{a} | | | | | Effects, P < | | |
|---|---|---|---|---|---|---|---|---|
| Activity^{b,c} | HC | HT | LC | LT | SEM | pH | Enzyme | pH × Enzyme |
| XY | 637.1 | 739.5 | 579.7 | 761.0 | 54.48 | 0.66 | 0.005 | 0.34 |
| END | 85.2 | 133.1 | 78.8 | 91.6 | 13.07 | 0.04 | 0.02 | 0.12 |
| EXO | 0.80 | 2.03 | 0.77 | 0.78 | 0.355 | 0.10 | 0.11 | 0.12 |
| GPY | 3.75 | 6.19 | 5.61 | 5.70 | 0.676 | 0.28 | 0.06 | 0.08 |
| XPY | 0.63 | 1.29 | 0.33 | 0.10 | 0.104 | <0.001 | 0.01 | 0.01 |
| PROT | 1.12 | 3.99 | 1.29 | 11.13 | 0.544 | <0.001 | <0.001 | <0.001 |
| AF | 4.60 | 7.70 | 5.87 | 5.83 | 1.088 | 0.78 | 0.18 | 0.17 |

^{a}HC = high pH with control TMR; HT = high pH with TMR treated with enzymes; LC = low pH with control TMR; LT = low pH with TMR treated with enzymes.
^{b}XY = xylanase; END = endoglucanase; EXO = exoglucanase; GPY = β-D-glucosidase; XPY = β-D-xylosidase; PROT = protease; AF = α-L-arabinofuranosidase.
^{c}XY and END are expressed as nmol xylose or glucose min⁻¹ mL⁻¹; EXO, GPY, XPY, and AF are expressed as nmol p-nitrophenol min⁻¹ mL⁻¹; PROT is expressed as the equivalent to the absorbance measured from the action of 1 μg of a standard protease (from S. griseus) under identical experimental conditions.

TABLE 14
Effects of pH and enzyme addition on DM, OM, fiber and starch digestion in
continuous culture

| | Treatment^{a} | | | | | Effects, P< | | |
|---|---|---|---|---|---|---|---|---|
| Digestion | HC | HT | LC | LT | SEM | pH | Enzyme | pH × Enzyme |
| Apparent, % | | | | | | | | |
| DM | 54.9 | 57.8 | 55.0 | 55.8 | 1.81 | 0.45 | 0.19 | 0.41 |
| OM | 56.2 | 59.3 | 55.4 | 56.3 | 1.80 | 0.20 | 0.17 | 0.43 |
| CP | 14.8 | 16.9 | 15.6 | 22.0 | 3.82 | 0.16 | 0.06 | 0.30 |
| True, % | | | | | | | | |
| DM | 66.2 | 69.2 | 64.9 | 64.6 | 1.52 | 0.07 | 0.36 | 0.26 |
| OM | 66.7 | 69.9 | 65.0 | 65.0 | 1.50 | 0.04 | 0.27 | 0.28 |
| CP | 56.2 | 59.3 | 54.9 | 59.3 | 3.12 | 0.81 | 0.17 | 0.80 |
| NDF | 23.6 | 33.8 | 16.7 | 21.1 | 4.07 | <0.001 | 0.004 | 0.12 |
| ADF | 28.4 | 34.7 | 14.7 | 14.3 | 5.23 | <0.001 | 0.20 | 0.16 |
| ADL | 17.7 | 24.4 | 19.0 | 20.7 | 5.79 | 0.78 | 0.35 | 0.57 |
| Hemicellulose | 18.3 | 32.8 | 18.7 | 28.2 | 3.41 | 0.22 | <0.001 | 0.16 |
| Cellulose | 30.5 | 36.7 | 14.2 | 13.2 | 5.22 | <0.001 | 0.32 | 0.18 |
| Starch | 91.8 | 93.1 | 93.0 | 93.5 | 13.93 | 0.57 | 0.53 | 0.82 |

^{a}HC = high pH with control TMR; HT = high pH with TMR treated with enzymes; LC = low pH with control TMR; LT = low pH with TMR treated with enzymes.

TABLE 15
Effects of pH and enzyme addition on VFA^{a} , lactic acid, and gas concentrations in
continuous culture

| | Treatment^{b} | | | | | Effects, P< | | |
|---|---|---|---|---|---|---|---|---|
| Item | HC | HT | LC | LT | SEM | pH | Enzyme | pH × enzyme |
| Total VFA, mM | 105.2 | 106.7 | 92.0 | 97.2 | 3.75 | 0.007 | 0.61 | 0.59 |
| BCVFA^{b}, mM | 2.63 | 2.83 | 0.84 | 0.91 | 0.419 | 0.001 | 0.74 | 0.89 |
| VFA, % | | | | | | | | |
| Acetate | 52.3 | 51.7 | 42.3 | 41.9 | 1.89 | <0.001 | 0.71 | 0.96 |
| Propionate | 26.3 | 23.4 | 37.5 | 38.9 | 1.47 | <0.001 | 0.62 | 0.17 |
| Butyrate | 13.9 | 17.9 | 11.2 | 8.9 | 1.93 | 0.005 | 0.53 | 0.07 |
| Iso-Butyrate | 0.72 | 0.56 | 0.42 | 0.51 | 0.078 | 0.002 | 0.57 | 0.08 |
| Valerate | 2.78 | 2.95 | 7.58 | 7.38 | 0.620 | <0.001 | 0.97 | 0.72 |
| Iso-Valerate | 1.83 | 2.08 | 0.49 | 0.46 | 0.391 | 0.003 | 0.77 | 0.72 |
| Caproate | 1.49 | 2.08 | 1.04 | 1.26 | 0.462 | 0.14 | 0.33 | 0.65 |
| Acetate: Propionate | 2.03 | 2.31 | 1.14 | 1.08 | 0.183 | <0.001 | 0.52 | 0.33 |
| Lactic acid, mM | 4.53 | 3.86 | 2.68 | 1.40 | 1.204 | 0.10 | 0.43 | 0.80 |
| Gas, % | | | | | | | | |
| CH₄ | 7.34 | 8.01 | 1.27 | 1.33 | 0.577 | <0.001 | 0.52 | 0.59 |
| CO₂ | 61.97 | 62.26 | 54.77 | 51.53 | 4.229 | 0.04 | 0.68 | 0.62 |

^{a}Values presented are averages of 4 determinations throughout the day (0, 2, 5, and 8 h post morning feed provision to the fermenters).
^{b}HC = high pH with control TMR; HT = high pH with TMR treated with enzymes; LC = low pH with control TMR; LT = low pH with TMR treated with enzymes.

TABLE 16
Effect of buffer pH and enzymes on the N metabolism of ruminal microbes in
continuous culture

| | Treatment^{a} | | | | | Effects, P< | | |
|---|---|---|---|---|---|---|---|---|
| | HC | HT | LC | LT | SEM | pH | Enzyme | pH × Enzyme |
| N—NH₃, mg/100 mL | 0.18 | 0.21 | 0.09 | 0.14 | 0.020 | 0.002 | 0.06 | 0.55 |
| N flow, g/d | | | | | | | | |
| Total | 1.50 | 1.46 | 1.48 | 1.37 | 0.055 | 0.14 | 0.07 | 0.33 |
| Ammonia | 0.005 | 0.006 | 0.003 | 0.004 | 0.001 | 0.002 | 0.05 | 0.52 |
| Non-ammonia | 1.50 | 1.46 | 1.48 | 1.36 | 0.055 | 0.15 | 0.07 | 0.32 |
| Bacterial | 0.72 | 0.74 | 0.69 | 0.65 | 0.061 | 0.29 | 0.86 | 0.67 |
| Dietary | 0.77 | 0.71 | 0.79 | 0.71 | 0.054 | 0.85 | 0.17 | 0.81 |
| EMPS^{b} | 30.2 | 34.7 | 27.2 | 25.9 | 3.12 | 0.09 | 0.61 | 0.38 |

^{a}HC = high pH with control TMR; HT = high pH with TMR treated with enzymes; LC = low pH with control TMR; LT = low pH with TMR treated with enzymes.
^{b}EMPS = Efficiency of microbial protein synthesis (g N/kg OM truly digested).

TABLE 17
Ingredients and chemical composition of the diets (DM basis)

| | Diet¹ | |
|---|---|---|
| Item | High Forage Control | Low Forage Control |
| Ingredient | (%) | |
| Barley silage | 44.5 | 18.2 |
| Alfalfa hay, chopped | 16 | 16 |
| Barley, steam rolled | 3.5 | 28 |
| Corn, dry rolled | 11.9 | 12.5 |
| Barley, ground¹ | 3.5 | 3.8 |
| Molasses beet¹ | 2.5 | 2.6 |
| Beet pulp, ground¹ | 1.2 | 1.3 |
| Alberta gold¹ | 3.5 | 3.6 |
| Soy pass¹ | 4.2 | 4.5 |
| Corn gluten meal¹ | 5 | 4.8 |
| Dicalcium phosphate¹ | 0.7 | 0.7 |
| Sodium bicarbonate¹ | 0.4 | 0.4 |
| Flavor¹ | 0.01 | 0.01 |
| Soybean oil¹ | 2.4 | 2.5 |
| Mineral and vitamin premix¹ | 1 | 1.1 |
| Chemical | (% of DM) | |
| Dry matter | 56.4 | 72.4 |
| Organic matter | 92 | 93.1 |
| Crude protein | 19.6 | 20.3 |
| Neutral detergent fiber | 23.9 | 21.9 |
| Acid detergent fiber | 12.4 | 10.3 |
| Starch | 26.2 | 31.6 |
| Net energy for lactation, | 1.62 | 1.78 |
| Mcal/kg² | | |

¹Ingredients that were in the pelleted supplement
²Based on values from NRC (2001)

TABLE 18
Dry matter intake and nutrient digestibility in the total tract of lactating
dairy cows fed high or low forage (F) diets with (+P) or without
(−P) protease supplementation

| Digesti- | Diet | | | | | | | |
|---|---|---|---|---|---|---|---|---|
| bility, | High Forage | | Low Forage | | | Significance of effect | | |
| % | −P | +P | −P | +P | SEM | F | P | F × P |
| Dry matter | 68.9^{ab} | 70.4^{c} | 68.0^{a} | 75.1^{d} | 1.3 | <0.01 | <0.01 | <0.01 |
| Organic | 69.7^{ab} | 71.2^{c} | 68.9^{a} | 75.4^{d} | 1.3 | <0.01 | <0.01 | <0.01 |
| matter | | | | | | | | |
| Nitrogen | 75.1^{b} | 78.0^{c} | 72.3^{a} | 80.3^{d} | 1.3 | NS² | <0.01 | <0.01 |
| Starch | 94.4^{a} | 97.1^{c} | 96.9^{b} | 96.4^{b} | 0.6 | <0.01 | <0.01 | <0.01 |
| ADF | 24.0^{a} | 26.5^{b} | 21.9^{a} | 29.6^{c} | 4.0 | NS | <0.01 | <0.01 |
| NDF | 34.4^{a} | 35.9^{a} | 35.3^{a} | 42.3^{b} | 2.9 | <0.01 | <0.01 | <0.01 |
| Hemi- | 45.6^{a} | 46.0^{a} | 50.0^{b} | 53.8^{c} | 2.1 | <0.01 | <0.01 | <0.01 |
| cellulose | | | | | | | | |

F = level of forage in the diet (high vs. low forage)
P = protease (non-protease vs. protease)
F × P = interaction between F and P.
NS = non-significant (P > 0.15).
DM = dry matter; NDF = neutral detergent fiber; ADF = acid detergent fiber
^{a,b,c}Means in the same row with different superscripts differ (P < 0.05).

TABLE 19
Enzymatic activities in strained ruminal fluid from lactating cows fed high or
low forage TMR diets without or with protease supplementation

| | Diet¹ | | | | | | | |
|---|---|---|---|---|---|---|---|---|
| | High Forage | | Low Forage | | | Significance of effect | | |
| Activity | −P | +P | −P | +P | SEM | F | P | F × P |
| XY | 672^{a} | 846^{b} | 744^{ab} | 1086^{c} | 72 | 0.05 | 0.02 | 0.01 |
| END | 296 | 460 | 368 | 480 | 63 | NS | <0.01 | NS |
| EXO | 39.5 | 39.7 | 42.7 | 34.2 | 4.6 | NS | NS | NS |
| GPY | 67.6 | 65.2 | 73.1 | 68.7 | 4.3 | NS | NS | NS |
| XPY | 33.0 | 33.1 | 33.4 | 28.0 | 7.5 | NS | NS | NS |
| PROT | 0.30^{a} | 0.31^{a} | 0.39^{a} | 0.74^{b} | 0.05 | <0.01 | <0.01 | <0.01 |
| AF | 56.1 | 60.1 | 67.7 | 67.7 | 7.4 | <0.01 | NS | NS |

F = level of forage in the diet (high vs. low forage),
P = protease (non-protease vs. protease), and
F × P = interaction between F and P.
XY = xylanase;
END = endoglucanase;
EXO = exoglucanase;
GPY = β-D-glucosidase;
XPY = β-D-xylosidase;
PROT = protease;
AF = α-L-arabinofuranosidase.
XY and END are expressed as nanomoles of xylose or glucose per minute per milliliter;
EXO, GPY, XPY, and AF are expressed as nanomoles of p-nitrophenol per minute per milliliter;
PROT is expressed as azocasein hydrolyzed per hour per milliliter.
NS = non-significant (P > 0.15).
^{a,b}Means in the same row with different superscripts differ (P < 0.05).

TABLE 20
Cumulative gas production (ml/g OM) profiles of alfalfa hay and
barley silage incubated with or without protease enzyme

| | Treatment | | | | | Significance | | |
|---|---|---|---|---|---|---|---|---|
| Time post inoculation | Alfalfa Hay | | Barley Silage | | | of effect | | |
| (h) | −P | +P | −P | +P | SEM | F | P | F × P |
| 2 | 13.4^{a} | 14.5^{b} | 19.3^{c} | 18.5^{c} | 0.4 | <0.01 | NS | 0.02 |
| 4 | 33.0^{a} | 36.8^{a} | 49.9^{b} | 50.1^{b} | 1.8 | <0.01 | NS | NS |
| 6 | 58.7^{a} | 63.8^{a} | 90.9^{b} | 93.6^{b} | 2.7 | <0.01 | 0.15 | NS³ |
| 12 | 150.6^{a} | 162.5^{b} | 208.8^{c} | 214.7^{c} | 4.0 | <0.01 | 0.04 | NS |
| 18 | 201.4^{a} | 219.5^{b} | 268.3^{c} | 275.0^{c} | 4.7 | <0.01 | 0.01 | NS |
| 24 | 241.9^{a} | 259.5^{b} | 317.5^{c} | 323.8^{c} | 5.0 | <0.01 | 0.02 | NS |
| 36 | 288.4^{a} | 305.1^{b} | 391.9^{c} | 396.0^{c} | 5.4 | <0.01 | 0.07 | NS |
| 48 | 312.3^{a} | 329.8^{b} | 428.2^{c} | 432.1^{c} | 5.6 | <0.01 | 0.07 | NS |

F = source of forage (alfalfa hay vs. barley silage),
P = protease (non-protease vs. protease), and
F × P = interaction between F and P.
³NS = non-significant (P > 0.15).
^{a,b,c}Means in the same row with different superscripts differ (P < 0.05).

1. A method of increasing digestibility of a forage or a grain feed comprising the steps of:
a) providing at least one protease;
b) providing a forage or a grain feed suitable for a ruminant animal;
c) applying the protease to the forage or the grain feed to form a feed composition; and
d) administering the composition to the animal, whereby an increase in digestibility is effected.

2. The method according to claim 1, wherein the forage or the grain feed is selected from the group consisting of alfalfa hay and silage, grass hay and silage, mixed hay and silage, straw, corn silage, corn grain, barley silage, barley grain, oilseeds or a combination thereof.

3. The method according to claim 1, wherein the forage is alfalfa forage or alfalfa-grass forage mixture.

4. The method according to claim 1, wherein the protease is derived from a bacterium or a fungus.

5. The method according to claim 0.4, wherein the bacterium is a species of the genus Bacillus.

6. The method according to claim 4, wherein the fungus is a species of the genus Trichoderma.

7. The method according to claim 4, wherein the protease is selected from the group consisting of a cysteine protease, a metalloprotease, an aspartate protease and a serine protease.

8. The method according to claim 7, wherein the protease is a serine protease.

9. The method according to claim 4, wherein the protease is subtilisin-like.

10. The method according to claim 4, wherein the protease is formulated as a solid, liquid, suspension, mineral block, salt, granule, pill, pellet or powder.

11. (Canceled)

12. The method according to claim 10, wherein the protease is in combination with one or more inert or active ingredients selected from the group consisting of carriers; diluents; flavorings; excipients; enzymes selected from the group consisting of cellulases, xylanases, glucanases, amylases and esterases; antibiotics; prebiotics; probiotics; micronutrients; vitamins; minerals and macronutrients.

13. The method according to claim 10, wherein the protease is applied to the forage or grain feed in an amount in the range of 0.1 to 20 mL/kg of dietary dry matter consumed.

14. The method according to claim 10, wherein the protease is applied to the forage or grain feed in an amount in the range of 0.5 to 2.5 mL/kg of dietary dry matter consumed.

15. The method according to claim 10, wherein the protease is applied to the forage or grain feed in an amount in the range of 0.75 to 1.5 mL/kg of dietary dry matter consumed.

16. The method according to claim 10, wherein the amount of protease comprises protease activity in the range of 1,000 to 23,000 protease units/kg dry matter as assayed at pH 6.0 and 39° C. using azocasein as substrate.

17. The method according to claim 10, wherein the amount of protease comprises protease activity in the range of 2,300 to 11,000 protease units/kg dry matter as assayed at pH 6.0 and 39° C. using azocasein as substrate.

18. The method according to claim 10, wherein the amount of protease comprises protease activity in the range of 3,300 to 6,800 protease units/kg dry matter as assayed at pH 6.0 and 39° C. using azocasein as substrate.

19-57. (Canceled)

58. A method of producing a feed additive comprising the steps of:
a) providing at least one protease;
b) mixing the protease with one or more inert or active ingredients to form the feed additive; and
c) feeding the feed additive to a ruminant animal or adding the feed additive to a forage or a grain feed for the animal, whereby an increase in digestibility is effected.

59. The method according to claim 58, wherein the forage or the grain feed is selected from the group consisting of alfalfa hay and silage, grass hay and silage, mixed hay and silage, straw, corn silage, corn grain, barley silage, barley grain, oilseeds or a combination thereof.

60. The method according to claim 58, wherein the forage is alfalfa forage or alfalfa-grass forage mixture.

61. The method according to claim 58, wherein the protease is derived from a bacterium or a fungus.

62. The method according to claim 61, wherein the bacterium is a species of the genus Bacillus.

63. The method according to claim 61, wherein the fungus is a species of the genus Trichoderma.

64. The method according to claim 61, wherein the protease is selected from the group consisting of a cysteine protease, a metalloprotease, an aspartate protease and a serine protease.

65. The method according to claim 64, wherein the protease is a serine protease.

66. The method according to claim 61, wherein the protease is subtilisin-like.

67. The method according to claim 61, wherein the protease is formulated as a solid, liquids, of suspension, mineral block, salt, granule, pill, pellet or powder.

68. (Canceled)

69. The method according to claim 67, wherein the protease is in combination with one or more inert or active ingredients selected from the group consisting of carriers; diluents; flavorings; excipients; enzymes selected from the group consisting of cellulases, xylanases, glucanases, amylases and esterases; antibiotics; prebiotics; probiotics; micronutrients; vitamins; minerals and macronutrients.

70. The method according to claim 67, wherein the protease is applied to the forage or grain feed in an amount in the range of 0.1 to 20 mL/kg of dietary dry matter consumed.

71. The method according to claim 67, wherein the protease is applied to the forage or grain feed in an amount in the range of 0.5 to 2.5 mL/kg of dietary dry matter consumed.

72. The method according to claim 67, wherein the protease is applied to the forage or grain feed in an amount in the range of 0.75 to 1.5 mL/kg of dietary dry matter consumed.

73. The method according to claim 67, wherein the amount of protease comprises protease activity in the range of 1,000 to 23,000 protease units/kg dry matter as assayed at pH 6.0 and 39° C. using azocasein as substrate.

74. The method according to claim 67, wherein the amount of protease comprises protease activity in the range of 2,300 to 11,000 protease units/kg dry matter as assayed at pH 6.0 and 39° C. using azocasein as substrate.

75. The method according to claim 67, wherein the amount of protease comprises protease activity in the range of 3,300 to 6,800 protease units/kg dry matter as assayed at pH 6.0 and 39° C. using azocasein as substrate.

76. (Canceled)

77. A method of producing a feed composition for feeding to a ruminant animal comprising the steps of:
a) providing at least one protease;
b) providing a forage or a grain feed; and
c) applying the protease to the forage or the grain feed to form the composition, whereby an increase in digestibility is effected.

78. The method according to claim 77, wherein the forage or the grain feed is selected from the group consisting of alfalfa hay and silage, grass hay and silage, mixed hay and silage, straw, corn silage, corn grain, barley silage, barley grain, oilseeds or a combination thereof.

79. The method according to claim 77, wherein the forage is alfalfa forage or alfalfa-grass forage mixture.

80. The method according to claim 77, wherein the protease is derived from a bacterium or a fungus.

81. The method according to claim 80, wherein the bacterium is a species of the genus Bacillus.

82. The method according to claim 80, wherein the fungus is a species of the genus Trichoderma.

83. The method according to claim 80, wherein the protease is selected from the group consisting of a cysteine protease, a metalloprotease, an aspartate protease and a serine protease.

84. The method according to claim 83, wherein the protease is a serine protease.

85. The method according to claim 80, wherein the protease is subtilisin-like.

86. The method according to claim 80, wherein the protease is formulated as a solid, liquid or suspension, mineral block, salt, granule, pill, pellet or powder.

87. (Canceled)

88. The method according to claim 86, wherein the protease is in combination with one or more inert or active ingredients selected from the group consisting of carriers; diluents; flavorings; excipients; enzymes selected from the group consisting of cellulases, xylanases, glucanases, amylases and esterases; antibiotics; prebiotics; probiotics; micronutrients; vitamins; minerals and macronutrients.

89. The method according to claim 86, wherein the protease is applied to the forage or grain feed in an amount in the range of 0.1 to 20 mL/kg of dietary dry matter consumed.

90. The method according to claim 86, wherein the protease is applied to the forage or grain feed in an amount in the range of 0.5 to 2.5 mL/kg of dietary dry matter consumed.

91. The method according to claim 86, wherein the protease is applied to the forage or grain feed in an amount in the range of 0.75 to 1.5 mL/kg of dietary dry matter consumed.

92. The method according to claim 86, wherein the amount of protease comprises protease activity in the range of 1,000 to 23,000 protease units/kg dry matter as assayed at pH 6.0 and 39° C. using azocasein as substrate.

93. The method according to claim 86, wherein the amount of protease comprises protease activity in the range of 2,300 to 11,000 protease units/kg dry matter as assayed at pH 6.0 and 39° C. using azocasein as substrate.

94. The method according to claim 86, wherein the amount of protease comprises protease activity in the range of 3,300 to 6,800 protease units/kg dry matter as assayed at pH 6.0 and 39° C. using azocasein as substrate.

95. (Canceled)

96. A feed additive comprising at least one feed-grade protease in combination with one or more feed-grade inert or active ingredients, wherein the protease is included in an amount which increases digestibility of a forage or feed grain when applied to the forage or the feed grain and fed to an animal.

97. The additive according to claim 96, wherein the protease is derived from a bacterium or a fungus, wherein the amount of protease is in the range of 100 to 500,000 units of protease per mL or gram in combination with the one or more feed-grade inert or active ingredients.

98. The additive according to claim 97, wherein the bacterium is a species of the genus Bacillus.

99. The additive according to claim 97, wherein the fungus is a species of the genus Trichoderma.

100. The additive according to claim 97, wherein the protease is selected from the group consisting of a cysteine protease, a metalloprotease, an aspartate protease and a serine protease.

101. The additive according to claim 100, wherein the protease is a serine protease.

102. The additive according to claim 97, wherein the protease is subtilisin-like.

103. The additive according to claim 97, wherein the one or more inert or active ingredients are selected from the group consisting of carriers; diluents; flavorings; excipients; enzymes selected from the group consisting of cellulases, xylanases, glucanases, amylases and esterases; antibiotics; prebiotics; probiotics; micronutrients; vitamins; minerals and macronutrients.

104. The additive according to claim 96, wherein the protease is present in the additive to yield an amount in the range of 0.1 to 20 mL/kg of dietary dry matter consumed, wherein the dry matter is a forage or a grain feed.

105. The additive according to claim 96, wherein the protease is present in the additive to yield an amount in the range of 0.5 to 2.5 mL/kg of dietary dry matter consumed, wherein the dry matter is a forage or a grain feed.

106. The additive according to claim 96, wherein the protease is present in the additive to yield an amount in the range of 0.75 to 1.5 mL/kg of dietary dry matter consumed, wherein the dry matter is a forage or a grain feed.

107. The additive according to claim 96, wherein the protease is present in an amount to yield a protease activity in the range of 1,000 to 23,000 protease units/kg dry matter when applied to a forage or a grain feed as assayed at pH 6.0 and 39° C. using azocasein as substrate.

108. The additive according to claim 96, wherein the protease is present in an amount to yield a protease activity in the range of 2,300 to 11,000 protease units/kg dry matter when applied to a forage or a grain feed as assayed at pH 6.0 and 39° C. using azocasein as substrate.

109. The additive according to claim 96, wherein the protease is present in an amount to yield a protease activity in the range of 3,300 to 6,800 protease units/kg dry matter when applied to a forage or a grain feed as assayed at pH 6.0 and 39° C. using azocasein as substrate.

110. (Canceled)

111. The additive according to claim 96, wherein the forage or the grain feed is selected from the group consisting of alfalfa hay and silage, grass hay and silage, mixed hay and silage, straw, corn silage, corn grain, barley silage, barley grain, oilseeds or a combination thereof.

112. The additive according to claim 111, wherein the forage is alfalfa forage or alfalfa-grass forage mixture.

113. A feed composition for feeding to a ruminant animal comprising a forage or a grain feed in combination with at least one protease, whereby an increase in digestibility is effected.

114. The composition according to claim 113, wherein the forage or the grain feed is selected from the group consisting of alfalfa hay and silage, grass hay and silage, mixed hay and silage, straw, corn silage, corn grain, barley silage, barley grain, oilseeds or a combination thereof.

115. The composition according to claim 113, wherein the forage is alfalfa forage or alfalfa-grass forage mixture.

116. The composition according to claim 113, wherein the protease is derived from a bacterium or a fungus.

117. The composition according to claim 116, wherein the bacterium is a species of the genus Bacillus.

118. The composition according to claim 116, wherein the fungus is a species of the genus Trichoderma.

119. The composition according to claim 116, wherein the protease is selected from the group consisting of a cysteine protease, a metalloprotease, an aspartate protease and a serine protease.

120. The composition according to claim 119, wherein the protease is a serine protease.

121. The composition according to claim 116, wherein the protease is subtilisin-like.

122. The composition according to claim 116, wherein the protease is formulated as a solid, liquid or suspension, mineral block, salt, granule, pill, pellet or powder.

123. (Canceled)

124. The composition according to claim 122, wherein the protease is in combination with one or more inert or active ingredients selected from the group consisting of carriers; diluents; flavorings; excipients; enzymes selected from the group consisting of cellulases, xylanases, glucanases, amylases and esterases; antibiotics; prebiotics; probiotics; micronutrients; vitamins; minerals and macronutrients.

125. The composition according to claim 122, wherein the protease is applied to the forage or the grain feed in an amount in the range of 0.1 to 20 mL/kg of dry matter consumed.

126. The composition according to claim 122, wherein the protease is applied to the forage or the grain feed in an amount in the range of 0.5 to 2.5 mL/kg of dry matter consumed.

127. The composition according to claim 122, wherein the protease is applied to the forage or the grain feed in an amount in the range of 0.75 to 1.5 mL/kg of dry matter consumed.

128. The composition according to claim 122, wherein the amount of protease comprises protease activity in the range of 1,000 to 23,000 protease units/kg dry matter as assayed at pH 6.0 and 39° C. using azocasein as substrate.

129. The composition according to claim 122, wherein the amount of protease comprises protease activity in the range of 2,300 to 11,000 protease units/kg dry matter as assayed at pH 6.0 and 39° C. using azocasein as substrate.

130. The composition according to claim 122, wherein the amount of protease comprises protease activity in the range of 3,300 to 6,800 protease units/kg dry matter as assayed at pH 6.0 and 39° C. using azocasein as substrate.

131-188. (Canceled)
